(19) United States
(12) Patent Application Publication
(10) Pub. No.: US 2026/0314948 A1
(43) Pub. Date: Oct. 15, 2026

(54) RADAR APPARATUS, RADAR SIGNAL PROCESSING METHOD, VITAL DETECTION APPARATUS, AND RADAR SIGNAL PROCESSING CIRCUITRY

(71) Applicant: Panasonic Automotive Systems Co., Ltd., Kanagawa (JP)

(72) Inventors: Takaaki KISHIGAMI, Tokyo (JP); Tomohiro YUI, Kanagawa (JP)

(73) Assignee: Panasonic Automotive Systems Co., Ltd., Kanagawa (JP)

(21) Appl. No.: 19/465,758

(22) Filed: Jan. 30, 2026

(30) Foreign Application Priority Data
Mar. 14, 2025 (JP) 2025-041394

(51) Int. Cl. A61B 5/0507; G01S 7/02

(52) U.S. Cl. CPC A61B 5/0507; G01S 7/0235

(57) ABSTRACT

The detection accuracy of vital information in a radar apparatus is improved. The radar apparatus includes control circuitry, which, in operation, controls transmission of a signal by using a first frame and a second frame different from the first frame; and a transmission antenna that transmits the signal. In the radar apparatus, a frame interval of the second frame is shorter than a frame interval of the first frame; a transmission period of the signal in the first frame is shorter than the frame interval of the second frame; the number of transmissions of the signal in the first frame is larger than the number of transmissions of the signal in the second frame; and the first frame includes a transmission duration in which the signal is transmitted and a non-transmission duration in which the signal is not transmitted, and the transmission duration is shorter than the non-transmission duration.

### TECHNICAL FIELD

The present disclosure relates to a radar apparatus, a radar signal processing method, a vital detection apparatus, and radar signal processing circuitry.

### BACKGROUND ART

Radar apparatuses that use radar transmission signals with short wavelengths, including microwaves or millimeter-waves that realize high resolution, have been under study in recent years.

In the position measurement using a radar apparatus, for example, a Doppler frequency detection is performed to detect the distance of a target object (or a target) or a relative speed between the radar apparatus and a target object. In addition, in the radar apparatus, it is also possible to measure (for example, detection or angle measurement) the azimuth angle or elevation angle of a target object by using a plurality of antennas.

As a configuration of the radar apparatus for performing the angle measurement processing, the following is used: a method (angle-of-arrival estimation method, direction-of-arrival (DOA) estimation) of estimating the angle of arrival (arrival direction) of a reflected wave from a target object based on a reception phase difference with respect to an antenna interval by receiving reflected waves of radar transmission signals from a target object by a plurality of antennas (an array antenna). For example, examples of the angle-of-arrival estimation method include a Fourier method and, as methods that can obtain high resolution, a Capon method, a multiple signal classification (MUSIC), and an estimation of signal parameters via rotational invariance techniques (ESPRIT).

In addition, as the radar apparatus, for example, the following has been proposed: a configuration (also referred to as a multiple input multiple output (MIMO) radar) in which a plurality of antennas (array antennas) are provided not only in a receiver but also in a transmitter, and beam scanning is performed by signal processing using the transmission and reception array antennas (for example, see NPL 1).

### CITATION LIST

### Patent Literature

### PTL 1

US Patent Application Publication No. 2019/0227156

### PTL 2

Japanese Patent Application Laid-Open No. 2020-148754

### PTL 3

US Patent Application Publication No. 2023/0003870

### PTL 4

Japanese Patent Application Laid-Open No. 2014-119344

### Non Patent Literature

### NPL 1

J. Li, and P. Stoica, “MIMO Radar with Colocated Antennas”, Signal Processing Magazine, IEEE Vol. 24, Issue: 5, pp. 106-114, 2007

### NPL 2

U. R. Acharya, K. P. Joseph, N. Kannathal, C. M. Lim, and J. S. Suri, “Heart rate variability: a review”, Medical and Biological Engineering and Computing Vol. 44, pp. 1031-1051, 2006

### NPL 3

F. Michler, B. Scheiner, T. Reissland, R. Weigel, and A. Koelpin, “Micrometer Sensing With Microwaves: Precise Radar Systems for Innovative Measurement Applications”, IEEE Journal of Microwaves, vol. 1, no. 1, pp. 202-217, 2021

### NPL 4

O. Kwon, J. Jeong, H. B. Kim, I. H. Kwon, S. Y Park, J. E. Kim, and Y Choi, “Electrocardiogram Sampling Frequency Range Acceptable for Heart Rate Variability Analysis”, Healthcare Informatics Research, Vol. 24, No. 3, July 2018, pp. 198-206, 2018

### NPL 5

M. Kronauge, H. Rohling, “Fast two-dimensional CFAR procedure”, IEEE Transactions on Aerospace and Electronic Systems, vol. 49, no. 3, pp. 1817-1823, 2013

### NPL 6

J. A. Cadzow, “Direction-of-arrival estimation using signal subspace modeling”, IEEE Transactions on Aerospace and Electronic Systems, vol. 28, no. 1, pp. 64-79, 1992

### NPL 7

A. Mukhtar, L. Xia, and T. B. Tang, “Vehicle Detection Techniques for Collision Avoidance Systems: A Review”, IEEE Transactions on Intelligent Transportation Systems, vol. 16, no. 5, pp. 2318-2338, 2015

### SUMMARY OF INVENTION

However, there is room for further study on a method for improving the detection accuracy of vital information in the radar apparatus (for example, a MIMO radar).

The non-limiting embodiments of the present disclosure contribute to providing a radar apparatus, a radar signal processing method, a vital detection apparatus, and radar signal processing circuitry each capable of improving the detection accuracy of the vital information in the radar apparatus.

A radar apparatus according to an exemplary embodiment of the present disclosure includes: control circuitry, which, in operation, controls transmission of a signal by using a first frame and a second frame different from the first frame; and
a transmission antenna that transmits the signal, in which
   a frame interval of the second frame is shorter than a frame interval of the first frame, a transmission period of the signal in the first frame is shorter than the frame interval of the second frame, the number of transmissions of the signal in the first frame is larger than the number of transmissions of the signal in the second frame, and the first frame includes a transmission duration in which the signal is transmitted and a non-transmission duration in which the signal is not transmitted, and the transmission duration is shorter than the non-transmission duration.

Note that general or specific embodiments may be implemented as a system, an apparatus, a method, an integrated circuit, a computer program or a storage medium, or any selective combination of the system, the apparatus, the method, the integrated circuit, the computer program, and the storage medium.

According to one embodiment of the present disclosure, the detection accuracy of the vital information in the radar apparatus can be improved.

Additional benefits and advantages of the disclosed exemplary embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF DRAWINGS

FIG. 1 illustrates a configuration example of a radar apparatus;

FIG. 2 is a block diagram illustrating a configuration example of the radar apparatus;

FIG. 3 illustrates an example of chirp signals;

FIG. 4 illustrates an example of chirp signals;

FIG. 5 illustrates an example of radar transmission signals;

FIG. 6 illustrates an example of the radar transmission signals;

FIG. 7 illustrates an example of transmission and reception chirp signals;

FIG. 8 is a block diagram illustrating a configuration example of the radar apparatus;

FIG. 9 is a block diagram illustrating a configuration example of the radar apparatus;

FIG. 10 is a block diagram illustrating a configuration example of the radar apparatus;

FIG. 11 illustrates an example of radar transmission signals;

FIG. 12 illustrates an example of radar transmission signals; and

FIG. 13 illustrates an example of radar transmission signals.

### DESCRIPTION OF EMBODIMENTS

A MIMO radar transmits, for example, signals (also referred to as radar transmission waves, radar signals, or radar transmission signals) multiplexed by using time division multiplexing (TDM), frequency division multiplexing (FDM), or code division multiplexing (CDM) from a plurality of transmission antennas. In addition, a MIMO radar receives, for example, signals (also referred to as radar reflected waves or reflected wave signals) reflected from peripheral objects using a plurality of reception antennas, and separates and receives the multiplexed transmission signals from the reception signals. With such processing, the MIMO radar can extract a propagation path response indicated by the product of the number of transmission antennas and the number of reception antennas, and can perform array signal processing on these reception signals as a virtual reception array.

In addition, in the MIMO radar, by appropriately disposing an element interval in the transmission and reception array antenna, an antenna aperture can be virtually enlarged, and an angle resolution can be improved (for example, see NPL 1).

In addition, radar apparatuses using radar transmission signals having short wavelengths such as microwaves or millimeter waves are becoming smaller and cheaper, and their range of applications is expanding, with studies underway to apply them to applications such as non-contact interfaces such as gesture detection, biological detection, and detection of vital information (also referred to as a vital sign or biological information) of a living body (for example, a human body), such as breathing or a heartbeat. For example, the application of a radar apparatus to the detection of vital information such as breathing or a heartbeat has the advantage of being non-contact and enabling real-time measurement without the subject being aware of the apparatus, and thus technological development therefor is progressing.

In addition, the variation status of the heartbeat is also referred to as heart rate variability (HRV), and has been reported to be related to the subject's autonomic nervous system activity, such as their state of relaxation or tension (for example, see NPL 2). For example, HRV indicates the balance between the sympathetic and the parasympathetic nerve systems, and a high HRV indicates that the parasympathetic nerve system is dominant and is associated with a state of low stress. The heartbeat detection enables estimation of such health or stress states, and is expected to have various applications.

There is room for, for example, improvement in the detection accuracy (or the position measurement accuracy) of a radar apparatus in the detection of the vital information.

The processing of detecting vital information such as breathing or a heartbeat using a radar apparatus may use, for example, a principle of detecting a change in the body surface position of a human body due to breathing or a heartbeat as a change in the reception phase (for example, see NPL 3).

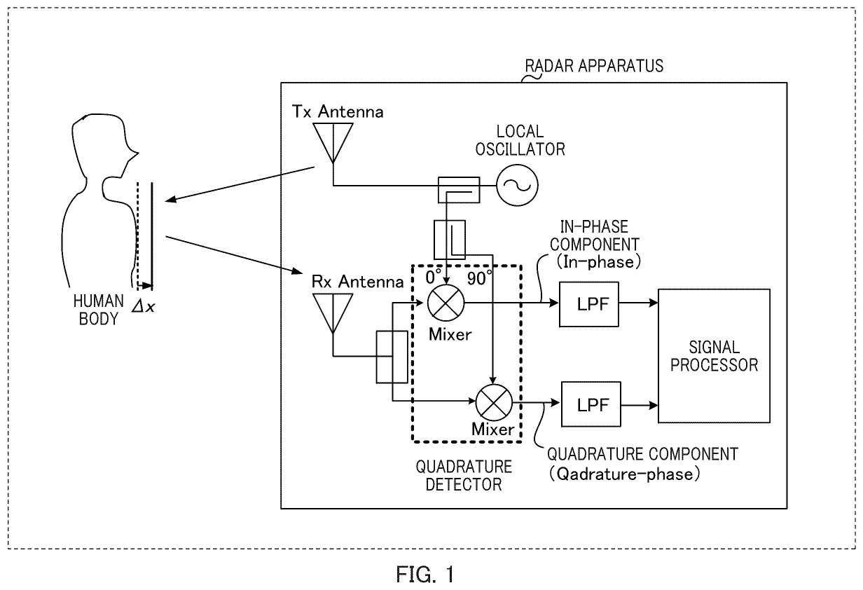

For example, as illustrated in FIG. 1, when a radar apparatus transmits a radar transmission wave (for example, frequency chirp signal waves (FMCW: frequency modulated continuous waves) or a pulse modulated wave) to the front surface of the chest of a human body, and the body surface displacement Δx due to breathing or heartbeat occurs, the path of the radar reflected wave changes by 2Δx. With such a displacement Δx, the radar reception phase changes. The change (also referred to as a reception phase variation) ΔΦ in the radar reception phase is represented by, for example, Expression (1). As shown in Expression (1), as the wavelength λ of the radar transmission wave becomes smaller, the change ΔΦ in the reception phase becomes larger, making it easier for the radar apparatus to easily detect a minute phase change.

$$\begin{matrix}( Expression 1 ) & \\ Δϕ = \frac{2 π}{λ} \times 2 Δ x = \frac{4 π}{λ} Δ x & [ 1 ]\end{matrix}$$

As an example, when the body surface displacement amount Δxᵣ due to breathing is 10 mm and a radar transmission wave having a frequency of 60 GHz is used, the wavelength λ of the radar transmission wave is 5 mm, and the reception phase variation amount ΔΦᵣ=8π [rad]. As described above, the radar apparatus can detect the number of breaths by detecting the phase variation in the complex phase plane to detect the reception phase variation and detecting a cycle of the reception phase variation due to breathing.

When the phase variation amount |ΔΦ| exceeds π, the radar apparatus may perform, for example, phase unwrapping processing in order to correctly estimate the phase variation amount (for example, see NPL 3). In addition, the center of the phase change may be observed to be shifted from the origin of the complex phase plane due to the influence of a stationary object or the like around a target object. In this case, the radar apparatus may, for example, observe the reception phase variation amount after adding a correction of setting the center of the phase change to the origin of the complex phase plane. For example, based on the fact that the phase change occurs on the circumference (or arc) of the complex phase plane, the center of the circle (or arc) is estimated and corrected so that the center of the circle (or arc) becomes the origin of the complex phase plane (for example, see NPL 3).

On the other hand, the body surface displacement Δxₕ due to the heartbeat is minute, namely approximately 1/100 of the body surface displacement Δxᵣ due to breathing. As an example, when the body surface displacement Δxₕ due to heartbeat is 0.1 mm and a radar transmission wave having a frequency of 60 GHz is used, the reception phase variation amount ΔΦₕ is 0.08π [rad]≈0.25 [rad].

In the radar apparatus, the reception phase variations (for example, ΔΦᵣ and ΔΦₕ) due to such breathing and heartbeat are observed in a superimposed manner. In order to separate the reception phase variation due to the breathing and the reception phase variation due to the heartbeat, the radar apparatus may utilize the difference between a time variation cycle of breathing and a time variation cycle of heartbeat, and apply frequency analysis processing using a band-pass filter or a Fourier transform (for example, a fast Fourier transform (FFT)) to separate and extract a breathing variability component and a heart rate variability component. Based on these components, the radar apparatus may perform the breathing detection (for example, the number of breaths) and the heartbeat detection (for example, the number of heartbeats, the HRV).

When a human body is stationary, biological detection such as breathing detection and heartbeat detection using a radar is subject to little disturbance due to a body movement component and can be performed with relatively satisfactory accuracy. On the other hand, when biological detection is performed in a situation including walking or body movement, the phase variation due to the walking or the body movement can be included as a disturbance component. Therefore, when it is difficult to appropriately remove the disturbance component, the accuracy of the biological detection may deteriorate. In addition, when the human body position changes as in walking, human body position detection and processing (tracking processing) that follows the displacement of the human body position are expected.

For example, in the biological detection in a vehicle cabin, a seated state can be regarded as a substantially stationary state, and an occupant entering or exiting the vehicle cabin or a driving the vehicle can be regarded as a walking or body moving state (also referred to as a walking/body moving state). For example, in the case where there are a plurality of occupants, the radar apparatus may perform the biological detection for each occupant. In addition, for example, in the biological detection in a bathroom, a state in a bathtub can be regarded as a substantially stationary state, and moving around the bathroom or taking a shower can be regarded as a walking/body moving state.

As a technology that enables biological detection by a radar apparatus even in such a stationary state and a state including a walking/body moving state, there is a method for transmitting a radar transmission wave using a transmission frame as described below (for example, see PTL 1).

For example, the radar apparatus may perform large body movement detection such as walking using a “transmission frame 1” and perform biological detection with minute displacement using a “transmission frame 2”. For example, the transmission frame 1 may be a frame for walking/body movement detection (for example, a Macro-Doppler detection frame) having a short observation time and a short transmission interval. In addition, the transmission frame 2 may be a frame for biological detection (for example, a Micro-Doppler detection frame) having a longer observation time and a longer transmission interval than the transmission frame 1. In PTL 1, for example, a portion of transmission signals between frames for performing Macro-Doppler detection (for example, walking/body movement detection) is used for Micro-Doppler detection (for example, biological detection).

Here, the heartbeat detection (for example, the number of heartbeats, the heart rate variability), detects the minute body surface displacement of a human body, the detection is easily affected by the peripheral environment or the body movement. As the heartbeat detection, the number of heartbeats is calculated as an average value of the heartbeats for approximately 60 seconds, and the heart rate variability is calculated based on a heartbeat interval (for example, the interval (RRI, R-R interval) between heartbeat R waves). Therefore, in the heartbeat detection, it is expected to accurately detect the heartbeat interval, and there is room for improvement in the accuracy of the non-contact heartbeat detection using the radar apparatus. The RRI represents a time interval of a heartbeat between an R wave and the next R wave, and the R wave is the most distinct waveform in the electrocardiogram and indicates ventricular contraction.

For example, in NPL 4, an optimal sampling frequency for an electrocardiogram (ECG) used for analyzing a heart rate variability (HRV) is studied. NPL 4 discloses that a sampling frequency of approximately 100 to 250 Hz or more is desirable in order to ensure the RRI detection accuracy, and it is difficult to allow an analysis error with a lower sampling frequency. In NPL 4, it is assumed that the RRI detection by a radar has the same characteristics. For example, by shortening the transmission interval of radar transmission waves (for example, chirp signals), the radar apparatus can observe the body surface displacement of a human body at a shorter time interval, and the detection accuracy of the heartbeat interval (for example, the RRI) is improved, and thus it is expected to improve the detection accuracy of the heart rate variability (HRV).

However, in PTL 1, a part of transmission signals between frames for Macro-Doppler detection (walking/body movement detection) is used for Micro-Doppler detection (biological detection), and therefore, when the transmission interval of the radar transmission waves for Micro-Doppler detection (biological detection) is shortened, the frame interval for Macro-Doppler detection (walking/body movement detection) is also shortened. Therefore, the transmission frequency of a radar transmission wave included in the frame for Macro-Doppler detection is increased, the transmission ON time becomes long, and the heat generation amount and the power consumption of the radar IC chip may become large.

On the other hand, when the frame interval for Macro-Doppler detection (for walking/body movement detection) is increased in order to reduce the transmission ON time and reduce the heat generation amount and the power consumption of the radar IC chip, it is difficult to shorten the transmission interval of the radar transmission waves for Micro-Doppler detection (for biological detection) to an interval equal to or less than the frame interval for Macro-Doppler detection (walking/body movement detection). As described above, in the method for increasing the frame interval for Macro-Doppler detection (for walking/body movement detection), it is difficult to shorten the transmission interval of the radar transmission waves in order to improve the detection accuracy of the heart rate variability.

The non-limiting embodiments of the present disclosure describe a method for increasing the transmission frequency of a transmission signal for heartbeat detection while reducing the transmission time of a walking/body movement detection signal of the living body in the radar apparatus in order to achieve both the improvement in the detection accuracy of the heart rate variability (HRV) and the reduction in the heat generation amount and the low power consumption of a radar IC chip.

In addition, in the non-limiting embodiments of the present disclosure, by reducing the transmission time of a walking/body movement detection signal of the living body, it is possible to ensure longer signal processing time and data transfer time in the signal processing of the walking/body movement detection of the living body, so that the hardware configuration of the digital signal processor (DSP) or the interface can be simplified to a configuration of a signal processing IC chip with a lower speed or an interface with a lower transmission rate, thereby reducing the cost of the radar apparatus.

In addition, in the non-limiting embodiments of the present disclosure, the reduction in the heat generation amount and the low power consumption of the radar IC chip can reduce the transmission ON time of a radar transmission wave, and therefore, it is also possible to reduce the mutual interference when another radar system is present in the vicinity. In addition, in the non-limiting embodiments of the present disclosure, it is possible to easily respond to the case where there is a regulation that sets the transmission ON time (for example, a duty ratio) of a radar transmission wave to a predetermined value or less. In addition, in the non-limiting embodiments of the present disclosure, it is expected that the amount of exposure to a human body would be reduced even in cases where the radar apparatus is expected to be in close proximity to the human body by reducing the ON time of a radar transmission wave.

Embodiments of the present disclosure will be described in detail with reference to the drawings. In the embodiments, the same constituent elements are identified with the same numerals, and a description thereof is omitted because of redundancy.

### [Configuration of Radar Apparatus]

The following describes a configuration (for example, a MIMO radar configuration) in which, in the radar apparatus, the radar transmitter transmits radar transmission signals from a plurality (for example, Nt) of transmission antennas, and the radar receiver separates and receives the radar transmission signals using a plurality (for example, Na) of reception antennas.

The radar apparatus is not limited to the MIMO radar configuration, and for example, the same operation can be performed even when the number of reception antennas is one (Na=1), and the same operation can be performed even when the number of transmission antennas is one (Nt=1). In the case of Nt=1, radar apparatus 1 described below does not need to perform MIMO multiplexing transmission processing and MIMO separation processing, and does not need to include transmission controller 105, phase rotator 106, switch (SW) 107, or output switch 209.

The following also describes, as an example, a configuration of a radar scheme (also referred to as, for example, chirp pulse transmission (fast chirp modulation)) that uses frequency-modulated pulse waves such as chirp pulses. Note that the modulation scheme is not limited to such a frequency modulation. For example, an exemplary embodiment of the present disclosure is also applicable to a radar scheme that uses a pulse compression radar configured to transmit a pulse train after performing phase modulation or amplitude modulation.

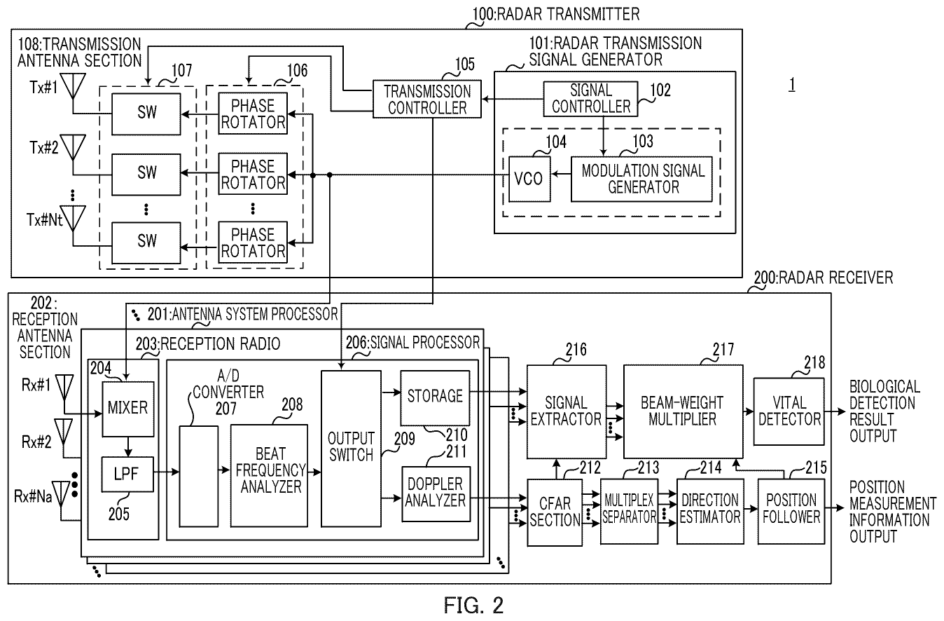

FIG. 2 illustrates a configuration example of radar apparatus 1 (for example, corresponding to a vital detection apparatus) according to the present embodiment.

Radar apparatus 1 of FIG. 2 includes, for example, radar transmitter (corresponding to a transmission branch or radar transmission circuitry) 100 and radar receiver (corresponding to a reception branch or radar reception circuitry) 200.

Radar transmitter 100 generates, for example, radar transmission signals and transmits the radar transmission signals at a predetermined transmission period using transmission antenna section 108.

Radar receiver 200 receives, for example, reflected wave signals—that are radar transmission signals reflected by a target object (target, not illustrated)—by using reception antenna section 202. Radar receiver 200 performs signal processing on the reflected wave signals received by reception antenna section 202. For example, radar receiver 200 may detect vital information based on the reflected wave signals after the signal processing.

### Configuration Example of Radar Transmitter 100

Radar transmitter 100 includes, for example, radar transmission signal generator 101, transmission controller 105, phase rotator 106, switch (SW) 107, and transmission antenna section 108.

Radar transmission signal generator 101 generates, for example, a radar transmission signal. The generated radar transmission signal may be, for example, a predetermined frequency-modulated wave (for example, a frequency chirp signal or a chirp signal).

Radar transmission signal generator 101 includes, for example, signal controller 102, modulation signal generator 103, and voltage controlled oscillator (VCO) 104. Hereinafter, each component in radar transmission signal generator 101 will be described.

Signal controller 102 controls, for example, the generation of radar transmission signals in modulation signal generator 103 or VCO 104. Signal controller 102 may output, for example, information related to the generation control of the radar transmission signals to modulation signal generator 103 and transmission controller 105.

Modulation signal generator 103 periodically generates, for example, a sawtooth-shaped modulation signal. Modulation signal generator 103 may generate, for example, the modulation signal based on information input from signal controller 102.

VCO 104 generates a chirp signal based on the modulation signal output from modulation signal generator 103, and outputs the chirp signal to phase rotator 106 and radar receiver 200 (mixer 204 described below).

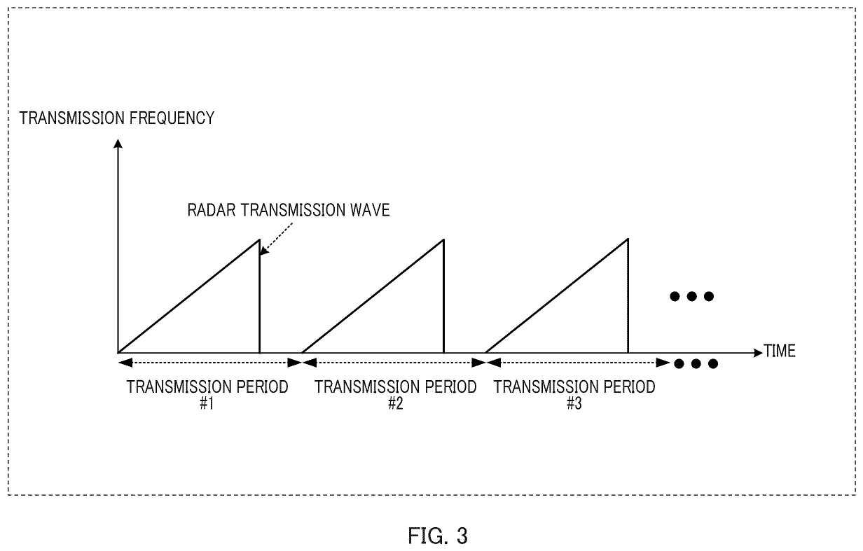

FIG. 3 illustrates an example of chirp signals transmitted from radar transmitter 100. For example, radar transmitter 100 transmits a plurality of chirp signals generated in radar transmission signal generator 101.

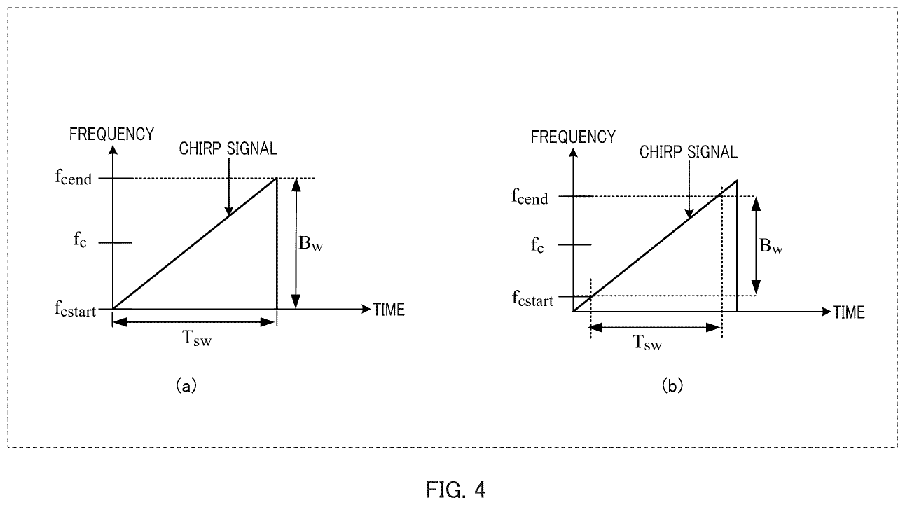

FIG. 4 illustrates an example of chirp signals output from radar transmission signal generator 101. As illustrated in FIG. 4, modulation parameters related to the chirp signals may include, for example, a center frequency f_{c}, a frequency sweep bandwidth B_{w}, a sweep start frequency f_{cstart}, a sweep end frequency f_{cend}, a frequency sweep time T_{sw}, and a frequency sweep change rate Dₘ. Note that Dₘ=B_{w}/T_{sw}. In addition, B_{w}=f_{cend}−f_{cstart} and f_{c}=(f_{cstart}+f_{cend})/2. In addition, the frequency sweep time T_{sw} corresponds to, for example, a time range (or referred to as a range gate) in which A/D sample data is taken in A/D converter 207 of radar receiver 200 described below. The frequency sweep time T_{sw} may be set in an entire section of the chirp signal as illustrated in part (a) in FIG. 4, for example, or may be set in a part of the section of the chirp signal as illustrated in part (b) in FIG. 4.

Note that FIGS. 3 and 4 illustrate an example of waveforms of an up-chirp in which the modulation frequency gradually increases with the passage of time, but the present disclosure is not limited thereto, and a down-chirp in which the modulation frequency gradually decreases with the passage of time may be applied. The same effect can be obtained regardless of whether the modulation frequency is an up-chirp or a down-chirp.

In FIG. 2, transmission antenna section 108 of radar transmitter 100 includes Nt transmission antennas (also represented by Tx #1 to Tx #Nt), and each transmission antenna may be individually connected to corresponding SW 107. In addition, SWs 107 corresponding to respective transmission antennas may be individually connected to phase rotators 106.

Transmission controller 105 controls the transmission of a chirp signal. For example, transmission controller 105 controls phase rotator 106 and SW 107 based on a chirp signal generation timing output from signal controller 102.

The Nt phase rotators 106 perform, for each transmission period of chirp signals, a predetermined phase rotation on the chirp signal input from radar transmission signal generator 101 based on control (for example, a phase control signal) from transmission controller 105, and output the signal subjected to the phase rotation to SW 107 (an operation example will be described below).

The n-th SW 107 switches whether or not to output (or radiate), from the n-th transmission antenna, the output from the n-th phase rotator 106 based on the control (for example, an antenna switching control signal) sent from transmission controller 105 (an operation example will be described below). Here, n=1 to Nt. For example, when the n-th SW 107 is in the output ON state, the transmission signal input from the n-th phase rotator 106 is output from the n-th transmission antenna. On the other hand, when the n-th SW 107 is in the output OFF state, the transmission signal input from the n-th phase rotator 106 is not output from the n-th transmission antenna.

Therefore, an output signal from phase rotator 106 corresponding to SW 107 in the output ON state among the plurality of SWs 107 is amplified to a predetermined transmission power and is radiated into space from the transmission antenna corresponding to SW 107 in the output ON state.

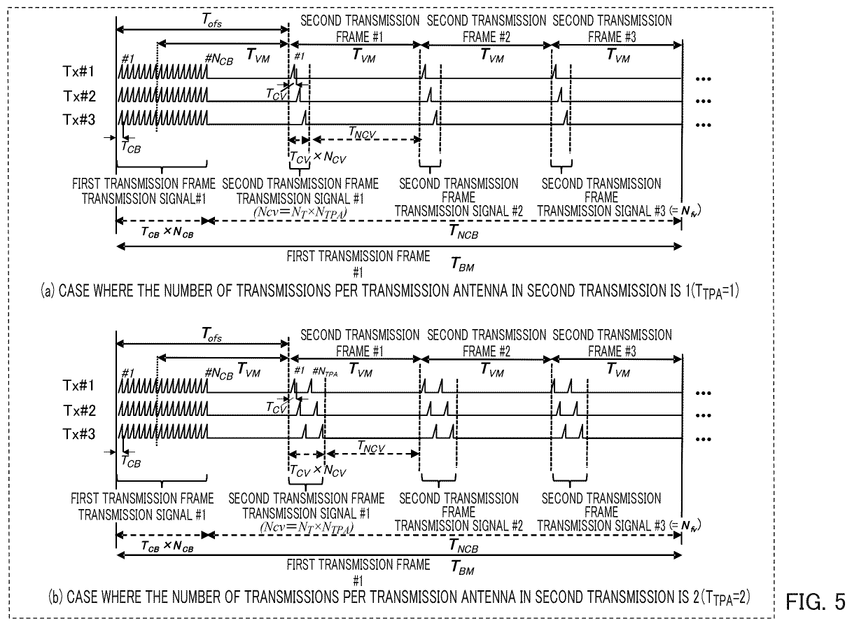

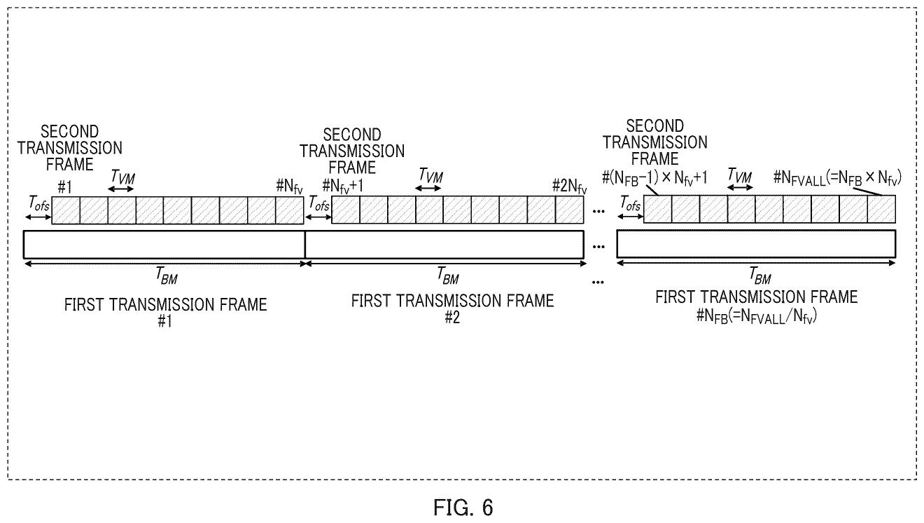

Hereinafter, an operation example of radar transmitter 100 will be described with reference to FIGS. 5 and 6. FIG. 5 illustrates an example of radar transmission signals when the number of transmission antennas Nt is 3 (Tx #1, Tx #2, and Tx #3). In FIG. 5, the vertical axis for each transmission antenna is a transmission frequency, and a sawtooth-shaped signal portion represents chirp signals. In addition, chirp signals transmitted simultaneously (Doppler multiplexing, code multiplexing) or time-divisionally from the transmission antennas indicate the case where the chirp signals having the same center frequency f_{c} and the same transmission bandwidth B_{w} are transmitted, but the transmitted chirp signals do not need to have the same center frequency f_{c} or the same transmission bandwidth B_{w}.

Signal controller 102 controls modulation signal generator 103 and VCO 104 to generate, for example, as illustrated in FIGS. 5 and 6, radar transmission signals (chirp signals) including a “first transmission frame” and a plurality (for example, N_{fv}) of “second transmission frames”. Note that FIG. 5 illustrates an example of N_{fv}=3, but the present disclosure is not limited thereto, and the value of N_{fv} may be another value.

The first transmission frame is, for example, a frame including a chirp transmission duration (=T_{CB}×N_{CB}) in which a chirp signal is transmitted N_{CB} times at a transmission period (T_{CB}) and a chirp non-transmission duration (=T_{NCB}). A frame length T_{BM} of the first transmission frame is T_{CB}×N_{CB}+T_{NCB}. For example, the number of first transmission frames may be more than one. When a plurality of first transmission frames are transmitted, the first transmission frames are transmitted at an interval T_{BM}.

The second transmission frame is, for example, a frame including a chirp transmission duration (=T_{CV}×N_{CV}) in which a chirp signal is transmitted N_{CV} times at a transmission period (T_{CV}) and a chirp non-transmission duration (=T_{NCV}). A frame length T_{VM} of the second transmission frame is T_{CV}×N_{CV}+T_{NCV}. The N_{fv} (e.g., two or more) second transmission frames are transmitted at a second transmission frame interval T_{VM}. In addition, the number of times the chirp signal is transmitted from each transmission antenna in the second transmission frame is set to “T_{TPA}”. Part (a) in FIG. 5 illustrates an example of T_{TPA}=1, and part (b) in FIG. 5 illustrates an example of T_{TPA}=2. Note that the value of T_{TPA} is not limited to 1 or 2, and may be another value.

In addition, as illustrated in FIG. 5, the transmission of the chirp signal of the second transmission frame is started after an offset period T_{ofs} from the start timing of the first chirp signal of the first transmission frame (hereinafter, T_{ofs} is referred to as a “second transmission frame offset period”). Here, by setting T_{ofs}>T_{CB}×N_{CB}, the transmission signal of the second transmission frame is repeatedly set N_{fV} (=floor[(T_{BM}−T_{ofs})/T_{VM}]) times in the chirp non-transmission duration of the first transmission frame. Here, floor(x) is a function that returns the largest integer value not exceeding a real number x.

Here, for example, “N_{FVAll}” indicates the number of second transmission frames with the unit of biological detection processing. For example, N_{FVAll}≥N_{fv}. In addition, for example, “N_{FB}” indicates the number of first transmission frames in the number of second transmission frames (N_{FVALL}) with the unit of biological detection processing. For example, N_{FB} may be set to ceil (N_{FVAll}/N_{fv}). FIG. 5 illustrates an example in which N_{FVAll}=N_{FB}×N_{fv}=3 and N_{FB}=1 are set.

In addition, in the first transmission frame, for example, simultaneous multiplexing transmission such as Doppler multiplexing (DDM) or code multiplexing (CDM) may be applied to the multiplexing transmission performed by the Nt (e.g., two or more) transmission antennas. Nt phase rotators 106 output, for example, signals subjected to a predetermined phase rotation based on phase control signals from transmission controller 105 to SWs 107 for each transmission period of the chirp signal as illustrated in FIG. 5.

For example, when Doppler multiplexing (DDM) is applied in the first transmission frame, transmission controller 105 may impart the following phase rotation to phase rotator 106 via a phase control signal.

Transmission controller 105 sets a phase rotation amount φ_{ndm} for imparting a Doppler shift amount DOP_{ndm}. Here, ndm=1 to N_{DM}. N_{DM} is the number of settings of different Doppler shift amounts, and is referred to as a “Doppler multiplexing number” below.

In radar apparatus 1, the Doppler multiplexing number N_{DM} may be set to the same number as the number Nt of transmission antennas used for the multiplexing transmission. Note that, in the following, the case where the Doppler multiplexing number N_{DM} is the same number as the number Nt of transmission antennas used for the multiplexing transmission (N_{DM}=Nt) will be described, but the present disclosure is not limited thereto. For example, when the multiplexing transmission is performed using a part of the transmission antennas, the Doppler multiplexing number N_{DM} may be set to a number smaller than Nt. Note that the Doppler multiplexing number N_{DM} is set to 2 or more.

The N_{DM} Doppler shift amounts DOP₁, DOP₂, . . . , and DOP_{N_DM} (“N_DM” is also referred to as “N_{DM}”) may be, for example, set to even intervals or may be set to uneven intervals. DOP₁, DOP₂, . . . , and DOP_{N_DM} may be set to satisfy, for example, 0≤DOP₁, DOP₂, . . . , and DOP_{N_DM}<1/T_{CB}. Alternatively, DOP₁, DOP₂, . . . , and DOP_{N_DM} may be set to satisfy, for example, Expression (2).

$$\begin{matrix}( Expression 2 ) & \\ \frac{- 1}{2 {T}_{CB}} \leq {DOP}_{1} , {DOP}_{2} , … , {DOP}_{N \_ DM} < \frac{1}{2 {T}_{CB}} & [ 2 ]\end{matrix}$$

In addition, for example, the minimum Doppler shift interval Δf_{MinInterval} among DOP₁, DOP₂, . . . , and DOP_{N_DM} may satisfy Expression (3). Note that the Doppler shift interval (also referred to as a Doppler multiplexing interval or a Doppler interval) may be defined as an absolute value of the difference between any two of Doppler shift amounts DOP₁, DOP₂, . . . , and DOP_{N_DM}.

$$\begin{matrix}( 3 ) & \\ 0 < Δ {f}_{MinInterval} \leq \frac{1}{{T}_{CB} {N}_{DM}} & [ 3 ]\end{matrix}$$

In addition, the phase rotation amount φ_{ndm} for imparting each of DOP₁, DOP₂, . . . , and DOP_{N_DM} may be assigned, for example, as in Expression (4).

(Expression 4)

φ_{ndm}=2πDOP_{ndm}T_{CB} [4]

Note that, when the Doppler shift amounts are set so that the Doppler shift interval is even interval Δf_{MinInterval} (hereinafter, referred to as “even interval Doppler shift amount setting”), the phase rotation amount φ_{ndm} for imparting DOP_{ndm} is assigned, for example, as in Expression (5).

(Expression 5)

φ_{ndm}=2π(ndm−1)Δf_{MinInterval}T_{CB} [5]

Here, as the minimum Doppler shift interval Δf_{MinInterval} is narrower, the interference between the DDM signals is more likely to occur, and the target detection accuracy is more likely to be reduced (for example, deteriorated). Therefore, for example, it is suitable to further expand the interval of the Doppler shift amounts within a range satisfying the constraint condition of Expression (3). For example, when the equality in Expression (3) is established (for example, Δf_{MinInterval}=1/(T_{CB}N_{DM})), the setting is made to maximally expand the interval in the Doppler domain between the DDM signals (hereinafter, referred to as “maximum even interval Doppler shift amount setting”). In this case, DOP₁, DOP₂, . . . , and DOP_{N_DM} are configured such that a phase rotation range of 0 or more and less than 2π is divided into N_{DM} equal parts, and different phase rotation amounts are assigned thereto. For example, the phase rotation amount φ_{ndm} for imparting DOP_{ndm} is assigned as in Expression (6). Note that, in the following, the angle is expressed in units of radians. For example, when the transmission antenna is set to Nt=2, the phase rotation amount φ_{ndm} for imparting DOP_{ndm} uses, for example, two-valued phase rotation amounts of 0 and π.

$$\begin{matrix}( Expression 6 ) & \\ {φ}_{ndm} = \frac{2 π ( ndm - 1 )}{{N}_{DM}} & [ 6 ]\end{matrix}$$

Note that the assignment of the phase rotation amounts for imparting DOP₁, DOP₂, . . . , and DOP_{N_DM} is not limited to such an assignment method. For example, the phase rotation amounts φ₁, φ₂, . . . , and φ_{N_DM} (where “N_DM” corresponds to N_{DM}) for DOP₁, DOP₂, . . . , and DOP_{N_DM} may be randomly assigned using a phase rotation amount assignment table.

In addition, in the even interval Doppler shift amount setting shown in Expression (5), the phase rotation amount may be set as in Expression (7) by setting Δf_{MinInterval}=1/(T_{CB}(N_{DM}+Nᵢₙₜ)). Here, Nᵢₙₜ takes an integer value. In this case, the Doppler shift amounts are set to be at uneven intervals. For example, when the transmission antennas are set to Nt=3 and N_{DM}=Nt and Nᵢₙₜ=1 are set, any three of four-valued phase rotation amounts of 0, π/2, π, and 3π/2 are set as the phase rotation amount φ_{ndm} for imparting DOP_{ndm}.

$$\begin{matrix}( Expression 7 ) & \\ ϕ = \frac{2 π ( ndm - 1 )}{{N}_{DM} + {N}_{int}} & [ 7 ]\end{matrix}$$

Transmission controller 105 outputs a phase control signal to phase rotator 106 to set the phase rotation amount φ_{ndm} for imparting the n_{dm}-th Doppler shift amount DOP_{ndm} in the m_{b}-th transmission period T_{CB} of the first transmission frame. Phase rotator 106 imparts the phase shift amount ψ_{ndm}(m_{b}) indicating the phase rotation amount for each transmission period shown in Expression (8) to an input chirp signal and outputs the chirp signal. Here, m_{b}=1 to N_{CB} and ndm=1 to N_{DM}.

(Expression 8)

ψ_{ndm}(m_{b})=(m_{b}−1)×φ_{ndm} [8]

The output (for example, referred to as a Doppler multiplexed signal (DDM signal)) from each of the Nt phase rotators 106 is amplified to a predetermined transmission power through SW 107 and is radiated into space from each of the Nt transmission antennas of transmission antenna section 108.

Here, transmission controller 105 outputs antenna switching control signals for setting the output ON of the Nt transmission antennas to SWs 107. SWs 107 simultaneously transmit the outputs from phase rotators 106 from the subsequent transmission antennas, based on the antenna switching control signals from transmission controller 105.

With the above operation, radar transmitter 100 can perform the simultaneous multiplexing transmission using DDM in the first transmission frame.

Note that, in order to reduce the power consumption during the simultaneous transmission, the transmission power of each transmission antenna in the first transmission frame may be reduced, for example, for each transmission antenna. For example, for performing the simultaneous multiplexing transmission using the Nt transmission antennas, radar transmitter 100 may reduce the transmission power of each transmission antenna to approximately 1/Nₜ of the transmission power required for outputting a signal from one transmission antenna and transmit the radar transmission signal. For example, transmission controller 105 may set the transmission power of each transmission antenna used for the simultaneous multiplexing transmission in the first transmission frame according to the number of transmission antennas used for the simultaneous multiplexing transmission. As a result, the transmission power for transmission signals in the first transmission frame can be reduced, and the power consumption of radar apparatus 1 can be reduced.

Note that the transmission power sum of all the transmission antennas for performing the simultaneous multiplexing transmission using the Nt transmission antennas in the first transmission frame may be the same as the transmission power from the transmission antennas for performing the time division multiplexing transmission of transmission signals in the second transmission frame. As a result, in the case of performing the heartbeat detection on a detection target object, the deterioration of the heartbeat detection performance is less likely to be affected even when radar transmitter 100 reduces the transmission power of each transmission antenna in the first transmission frame as described above (for example, to approximately 1/Nt) and performs the simultaneous multiplexing transmission.

In addition, for example, when code multiplexing (CDM) is applied in the first transmission frame, transmission controller 105 may impart the following phase rotation to phase rotator 106 via a phase control signal.

Hereinafter, a setting example of the phase rotation when an orthogonal code sequence is used as the code used in the code multiplexing (CDM) will be described.

For example, orthogonal code sequences consisting of N_{CM} code lengths Loc are represented by Code_{ncm}={OC_{ncm}(1), OC_{ncm}(2), . . . , and OC_{ncm}(Loc)}. OC_{ncm}(noc) represents an n_{oc}-th code element in the n_{cm}-th orthogonal code sequence Code_{ncm}. Here, noc is an index of the code element, and noc=1 to Loc. In addition, in radar apparatus 1, the number of code multiplexing may be set to the same number as the number Nt of transmission antennas used for the multiplexing transmission. Note that, for example, when the multiplexing transmission is performed using a part of the transmission antennas, the number of code multiplexing may be set to a number smaller than Nt. Note that the number of code multiplexing is set to 2 or more.

As the N_{CM} orthogonal code sequences, orthogonal code sequences of Nt or more transmission antennas that are used for the multiplexing transmission using the code multiplexing (CDM) in the first transmission frame may be used. The orthogonal code sequences are, for example, codes that are orthogonal (uncorrelated) to each other. For example, the orthogonal code sequences may be a Walsh-Hadamard codes. In this case, the code length L_{OC} for generating the N_{CM} orthogonal code sequences are represented by Expression (9).

(Expression 9)

Loc=2^{ceil[log}^{₂}^{(N}^{_{CM}}^{)]} [9]

Here, ceil[x] denotes the operator (ceiling function) that outputs a minimum integer greater than or equal to real number x.

For example, when N_{CM}=2, code length Loc of Walsh-Hadamard codes is equal to 2, and orthogonal code sequences are represented by Code₁={1, 1} and Code₂={1, −1}. When a code element in an orthogonal code sequence is 1, 1=exp(j0), with the phase thereof being 0. When a code element in an orthogonal code sequence is −1, −1=exp(jπ), with the phase thereof being π.

Further, for example, when N_{CM}=4, code length Loc is equal to 4, and orthogonal code sequences are represented by Code₁={1, 1, 1, 1}, Code₂={1, −1, 1, −1}, Code₃={1, 1, −1, −1}, and Code₄={1, −1, −1, 1}.

Note that, code elements in an orthogonal code sequence are not limited to real numbers and may include complex number values. For example, orthogonal code sequence Code_{ncm} as in Expression 10 may be used. Here, ncm=1, . . . , Nₜ. In this case, a code length used to generate orthogonal code sequences with the number of codes N_{CM} is represented by Loc=N_{CM}.

$$\begin{matrix}( Expression 10 ) & \\ {Code}_{ncm} = \{ 1 , exp [ j \frac{2 π}{{N}_{CM}} ( ncm - 1 ) ] , exp [ j \frac{2 π}{{N}_{CM}} 2 ( ncm - 1 ) ] , … , exp [ j \frac{2 π}{{N}_{CM}} ( {N}_{CM} - 1 ) ( ncm - 1 ) ] \} & [ 10 ]\end{matrix}$$

Transmission controller 105 outputs a phase control signal to set the phase shift amount ψ_{ncm}(m_{b}) for imparting the n_{cm}-th orthogonal code Code_{ncm} in the m_{b}-th transmission period T_{CB} of the first transmission frame. The phase rotator imparts the phase shift amount ψ_{ndm}(m_{b}) indicating the phase rotation amount shown in Expression (11) to an input chirp signal and outputs the chirp signal. Here, m_{b}=1 to N_{CB}.

(Expression 11)

ψ_{ncm}(m_{b})=angle[OC_{ncm}(mod(m_{b}−1,Loc)+1)] [11]

Next, in the second transmission frame, for example, multiplexing transmission using time division multiplexing (TDM) may be applied to the multiplexing transmission by the Nt (e.g., two or more) transmission antennas.

For example, transmission controller 105 outputs antenna switching control signals to SWs 107 to sequentially turn on the transmission antenna in a time division manner for each transmission period. Each SW 107 time-divisionally transmits, from the subsequent transmission antenna, the output from corresponding phase rotator 106 based on the antenna switching control signal.

For example, N_{TPA} chirp signals may be transmitted for each transmission antenna. In this case, Ncv (=Nt×N_{TPA}) chirp signals are output per second transmission frame. Here, N_{TPA} is the number of times the chirp signal is transmitted per transmission antenna, and a value of 1 or more is set for N_{TPA}. In addition, N_{TPA} is set to a smaller number than N_{cv}. For example, 1≤N_{TPA}<N_{cv}/Nt.

For example, part (a) in FIG. 5 illustrates the case where N_{TPA}=1 using the transmission antennas of Nt=3, and part (b) in FIG. 5 illustrates the case where N_{TPA}=2 using the transmission antennas of Nt=3. For example, in part (a) in FIG. 5, N_{cv}=3 (3×1) chirp signals are output per second transmission frame. In addition, for example, in part (b) in FIG. 5, N_{cv}=6 (3×2) chirp signals are transmitted per second transmission frame.

Hereinafter, an operation example in a case illustrated in FIG. 5 will be described.

Transmission controller 105 outputs an antenna switching control signal to SW 107 to output the chirp signal from the mod(mᵥ−1, Nt)+1-th transmission antenna in the mᵥ-th transmission period T_{CV} of the second transmission frame. Here, mod(x, y) is a modulo operator and is a function that outputs the remainder after dividing x by y.

In addition, transmission controller 105 outputs, for example, a phase control signal to each phase rotator 106 so that the phase shift amount is constant, for example, zero, such that the phase rotation amount φₙₜ=0. Phase rotator 106 sets in such a way that phase shift amount ψₙₜ(mᵥ)=0 for each transmission period T_{CV} and outputs the input chirp signal. Here, mᵥ=1 to N_{CV} and nt=1 to Nt.

SW 107 time-divisionally transmits, from the subsequent transmission antenna, the output from each phase rotator 106 based on the antenna switching control signal from transmission controller 105. For example, in the example of FIG. 5, in the first transmission period of the second transmission frame, the first transmission antenna (Tx #1) is set to the output ON state, and a chirp signal is output from the first transmission antenna. In addition, in the example of FIG. 5, in the second transmission period of the second transmission frame, the second transmission antenna (Tx #2) is set to the output ON state, and a chirp signal is output from the second transmission antenna. Similarly, in the example of FIG. 5, in the third transmission period of the second transmission frame, the third transmission antenna (Tx #3) is set to the output ON state, and a chirp signal is output from the third transmission antenna. Hereinafter, the operation of sequentially transmitting from the Nt transmission antennas N_{TPA} times (once in part (a) in FIG. 5 and twice in part (b) in FIG. 5) is performed.

After being amplified to a predetermined transmission power through the transmission antenna switching via SW 107, the signals are radiated into space from the transmission antennas of transmission antenna section 108.

With the above operation, the plurality of transmission antennas can perform the multiplexing transmission using TDM in the second transmission frame.

Note that, in the example of FIG. 5, the example has been described in which the MIMO multiplexing transmission is performed by setting the numbers of transmission antennas used in the first transmission frame and in the second transmission frame to be the same, but the present disclosure is not limited thereto. For example, the MIMO multiplexing transmission may be performed using different numbers of transmission antennas in the first transmission frame and in the second transmission frame.

In addition, for example, radar transmitter 100 may perform the MIMO multiplexing transmission using a smaller number of transmission antennas than Nt in the first transmission frame. In addition, for example, radar transmitter 100 may perform the MIMO multiplexing transmission using a smaller number of transmission antennas than Nt in the second transmission frame.

Radar apparatus 1 performs the walking/body movement detection and the biological detection described below using the transmission signals included in the first transmission frame and the second transmission frame described above (the examples will be described below in the description of the operation example of radar receiver 200).

Radar apparatus 1 performs the walking/body movement detection using transmission signals (for example, reflected wave signals reflected by a living body (for example, the human body)) of the first transmission frame, but the present disclosure is not limited thereto, and for example, the biological detection (breathing detection, heartbeat detection, and the like) may be performed using a part of the transmission signals of the first transmission frame. In addition, radar apparatus 1 performs the biological detection (detection of the vital information of the living body) using transmission signals (for example, reflected wave signals reflected by the living body) of the plurality of second transmission frames, but the present disclosure is not limited thereto, and for example, the walking/body movement detection may be performed using the transmission signals of the plurality of second transmission frames.

In radar apparatus 1, various effects are obtained by setting at least one of the transmission period, the number of transmissions, and the transmission interval as follows in consideration of the detection processing.

For example, the Doppler frequency caused by walking speed or body movement may fluctuate faster and be higher than the Doppler frequency due the breathing and heartbeat in the biological detection. Therefore, in radar apparatus 1, it is expected to increase the maximum Doppler frequency that can be detected in order to detect the Doppler frequency caused by walking speed or the body movement. For example, the chirp transmission period (T_{CB}) in the first transmission frame may be set to be shorter than the second transmission frame interval (T_{VM}) (T_{CB}<T_{VM}, for example, T_{CB} is approximately several 100 μs to 1 ms).

In addition, for example, the number of times N_{CB} the chirp signal is transmitted in the first transmission frame may be set to be larger than the number of chirp signal transmissions N_{CV} in the second transmission frame (N_{CV}<N_{CB}). With this setting, the observation time of the Doppler frequency using a chirp signal in the first transmission frame is increased, and the resolution of the Doppler frequency caused by the walking speed or the body movement is improved, so that the Doppler detection accuracy in radar apparatus 1 can be improved. In addition, for example, when there are a plurality of target objects, the separation detection performance in the Doppler frequency domain can be improved. The analysis of the Doppler frequency caused by the walking speed or the body movement described above is performed by Doppler analyzer 211 described below.

In addition, when the number of times N_{CB} the chirp signal is transmitted in the first transmission frame is set to be larger than the number of chirp signal transmissions N_{CV} in the second transmission frame (N_{CV}<N_{CB}), the following may occur: when time division multiplexing is used in a MIMO radar that uses a plurality of transmission antennas, a longer switching time per transmission antenna is required, which tends to lengthen the transmission ON time in the first transmission frame and the power consumption increases. Therefore, simultaneous multiplexing transmission such as Doppler multiplexing, code multiplexing, or a combination of Doppler multiplexing and code multiplexing may be applied to the transmission of chirp signals in the first transmission frame. As a result, the transmission ON time in the first transmission frame can be reduced to 1/(number of transmission antennas Nt) as compared with the time division multiplexing transmission.

In addition, for example, radar apparatus 1 can follow an assumed target object speed of the walking even when the movement distance is approximately 0.1 m. Therefore, for example, radar apparatus 1 can follow the position information of the walking target object even when the interval (T_{BM}) between the plurality of first transmission frames is approximately 100 ms with respect to the speed of approximately 1 m/s of the walking. On the other hand, the second transmission frame interval (T_{VM}) may be shortened to accurately detect the heart rate variability (HRV) in the biological detection (for example, may be approximately several 10 ms) as compared with T_{BM}. For example, the second transmission frame interval (T_{VM}) may be set to be shorter than the first transmission frame interval (T_{BM}) (T_{VM}<T_{BM}). The position information following processing of the walking target object or the like described above is performed by position follower 215 described below.

In addition, for example, the first transmission frame may include a chirp signal transmission duration (=T_{CB}×N_{CB}) and a chirp signal non-transmission duration (=T_{NCB}). For example, in the first transmission frame, by setting the transmission duration of the chirp signal to be shorter than the non-transmission duration of the chirp signal (for example, T_{CB}×N_{CB}<T_{NCB}), the transmission time is reduced, and the power consumption of radar apparatus 1 can be reduced. For example, when T_{BM}=100 ms, T_{CB}=100 μs, and N_{CB}=32 are set, the transmission duration of the chirp signal can be set to 10% or less of the first transmission frame length (T_{BM}), and radar apparatus 1 can be significantly reduced in power consumption.

In addition, for example, the number of chirp signal transmissions in the second transmission frame is N_{CV}, and N_{TPA} (for example, one or several) chirp signals may be transmitted for each transmission antenna. Here, when N_{TPA}>1, radar apparatus 1 may perform the walking/body movement detection based on the reception phase difference between the plurality of chirp signals for each antenna. As a result, for example, radar apparatus 1 can detect the abrupt change in the body movement, and can suppress the increase in the heartbeat detection error. In addition, for example, when an abrupt change in the body movement can be ignored, radar apparatus 1 can perform in-phase addition of the plurality of chirp signals for each antenna, and the effect of improving the reception quality (for example, signal to noise ratio (SNR)) by the coherent addition gain can be obtained. The walking/body movement detection or the in-phase addition processing using a transmission signal of the second transmission frame described above is performed by signal extractor 216 described below.

In addition, a chirp signal in the second transmission frame is transmitted N_{TPA} times (for example, one or several) for each transmission antenna, and therefore, it is more suitable that the time division multiplexing is applied in the MIMO radar using a plurality of transmission antennas as compared with the simultaneous multiplexing transmission to the chirp signal in the second transmission frame. By applying the time division multiplexing to a chirp signal in the second transmission frame, the following effects can be obtained.
1) When the simultaneous multiplexing transmission is applied, the radar signal transmission is performed by the number of transmissions in units of the minimum transmission period N_{demul} in order to separate multiplexed signals by the code multiplexing or the Doppler multiplexing. For example, the chirp signal is transmitted an integer multiple of N_{demul} times. For example, when the two-valued phase rotation (for example, [0, π]) is imparted in phase rotator 106 and the code multiplexing transmission is performed using the transmission antennas of Nt=3, an orthogonal code of the code length Loc≥4 or more is used. Therefore, the minimum transmission period N_{demul} is set to 4, and the chirp signal is transmitted at least four times. On the other hand, in the time division multiplexing using the transmission antennas of Nt=3, in the case of N_{TPA}=1, it is sufficient that the chirp signal is transmitted three times, thereby shortening the transmission time as compared with the case of the code multiplexing. As described above, the number of transmission antennas Nt used for the time division multiplexing transmission may be set to be smaller than the minimum transmission period (minimum number of transmissions) N_{demul} of the transmission signal in the simultaneous multiplexing transmission. When the two-valued phase rotation (for example, [0, π]) is imparted in phase rotator 106 and the number of transmission antennas Nt is not a power of 2, the time division multiplexing can shorten the transmission time as compared with the code multiplexing.
2) In the case of applying the simultaneous multiplexing transmission, when the transmission power setting (for example, the constraint) is used to set the total transmission power of the transmission antennas to be the same as the transmission power during the transmission by one antenna, the number of chirp signal transmissions is set to Nt×N_{TPA} in order to set the transmission power to be the same as the number Nt×N_{TPA} of chirp transmissions, in the time division multiplexing transmission. Here, when Nt×N_{TPA} is not an integer multiple of N_{demul} in the simultaneous multiplexing transmission, a larger number of chirp signal transmissions may be set. For example, in the case of Nt=3 and N_{TPA}=2, in the time division multiplexing transmission, the number of chirp signal transmissions is set to 6, while in the simultaneous multiplexing transmission, in the case of the minimum transmission period N_{demu}=4, the number of chirp signal transmissions is set to 8.

As described above, in the time division multiplexing transmission, the number of times a radar is transmitted can be further reduced as compared with the simultaneous multiplexing transmission, so that the transmission ON time of the second transmission frame can be reduced, and the power consumption can be reduced. In addition, the time division multiplexing transmission can easily perform the separation processing and can sufficiently suppress the mutual interference between the simultaneous multiplexed signals as compared with the simultaneous multiplexing transmission, and thus it is more suitable for detecting the minute phase variation.

In addition, the chirp transmission period T_{CB} in the first transmission frame may be set to be equal to or longer than the chirp transmission period T_{CV} in the second transmission frame. For example, the chirp transmission period T_{CB} in the first transmission frame and the chirp transmission period T_{CV} in the second transmission frame may be set to be the same. Alternatively, the chirp transmission period T_{CB} in the first transmission frame may be set to be larger than T_{CV} in order to further improve the Doppler frequency resolution (T_{CV}≤T_{CB}).

In addition, the second transmission frame may include a chirp signal transmission duration (=T_{CV}×N_{CV}) and a chirp signal non-transmission duration (=T_{NCV}). Here, in the second transmission frame, by setting the transmission duration of the chirp signal to be shorter than the non-transmission duration of the chirp signal (for example, T_{CV}×N_{CV}<T_{NCV}), the transmission time is reduced, and the power consumption of radar apparatus 1 can be reduced. For example, by setting T_{VM}=10 ms, T_{CV}=100 μs, N_{CV}=6, Nt=3, and N_{TPA}=2, the transmission duration of the chirp signal can be set to 10% or less of the second transmission frame length (T_{VM}), and radar apparatus 1 can be significantly reduced in power consumption.

### Configuration Example of Radar Receiver 200

In FIG. 2, radar receiver 200 includes reception antenna section 202 (for example, Rx #1 to Rx #Na). In addition, radar receiver 200 includes Na antenna system processors 201, constant false alarm rate (CFAR) section 212, multiplex separator 213, direction estimator 214, position follower 215 (for example, corresponding to the first detection circuitry), signal extractor 216, beam-weight multiplier 217, and vital detector 218 (for example, corresponding to the second detection circuitry). Here, Na≥1.

Antenna system processor 201 may be provided, for example, corresponding to each of the Na reception antennas.

Each of the Na reception antennas (Rx #1 to Rx #Na) receives a reflected wave signal that is a radar transmission signal transmitted from transmission antenna section 108 of radar transmitter 100 and reflected by a target object (for example, a reflective target object including a radar measurement target), and outputs the received reflected wave signal to the corresponding antenna system processor 201 as a reception signal.

Each antenna system processor 201 includes reception radio 203 and signal processor 206.

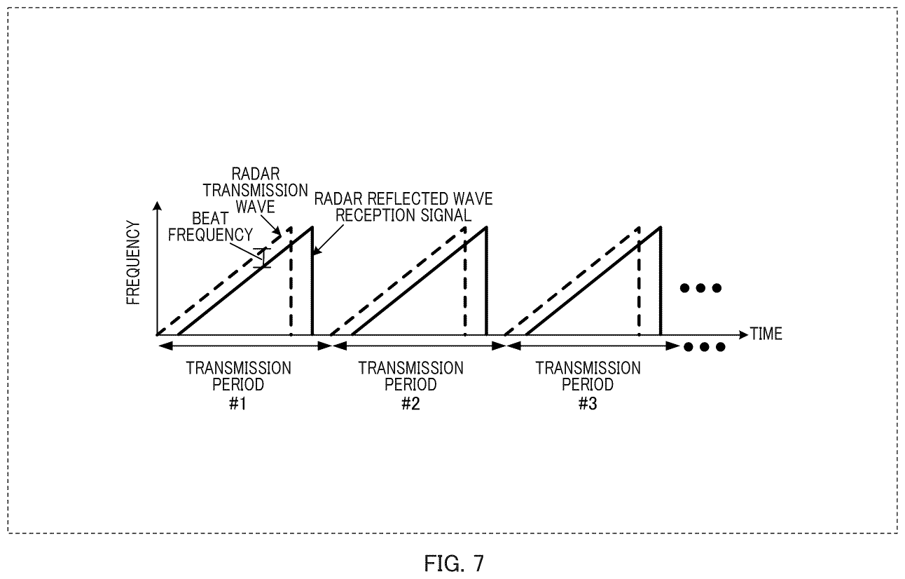

Reception radio 203 includes mixer 204 and low pass filter (LPF) 205. In reception radio 203, mixer 204 mixes a received reflected wave signal (reception signal) with a chirp signal that is the transmission signal. In addition, by allowing the output of mixer 204 to pass through LPF 205, a beat signal having a frequency corresponding to the delay time of the reflected wave signal is extracted. For example, as illustrated in FIG. 7, the difference frequency between the frequency of a transmission signal (transmission frequency-modulated wave) and the frequency of a reception signal (reception frequency-modulated wave) is obtained as a beat frequency (or a beat signal).

In FIG. 2, signal processor 206 of each antenna system processor 201-z (z=one of 1 to Na) includes A/D converter 207, beat frequency analyzer 208, output switch 209, storage 210, and Doppler analyzer 211.

Signals (for example, beat signals) output from LPF 205 are converted into discrete sample data that has been discretely sampled by A/D converter 207 in signal processor 206.

Beat frequency analyzer 208 performs FFT processing on N_{data1} pieces of discrete sample data obtained in a predetermined time range (range gate) T_{SW1} for each transmission period T_{CB} in the first transmission frame. In addition, beat frequency analyzer 208 performs FFT processing on N_{data2} pieces of discrete sample data obtained in a predetermined time range (range gate) T_{SW2} for each transmission period T_{CV} in the second transmission frame.

Note that a chirp signal of the first transmission frame and a chirp signal of the second transmission frame may be transmitted using the same modulation parameters (for example, the center frequency f_{c}, the frequency sweep bandwidth B_{w}, and the frequency sweep change rate Dₘ) (hereinafter, referred to as “identical chirp signals”) or may be transmitted using different modulation parameters (hereinafter, referred to as “different chirp signals”).

For example, when the identical chirp signals are used in the first transmission frame and the second transmission frame, the range gate T_{sw1} of the first transmission frame and the range gate T_{sw2} of the second transmission frame may be set to the same time range. As a result, the distance ranges that can be detected in the first transmission frame and the second transmission frame are the same, and the distance information represented by the distance index is also the same.

In addition, for example, when the different chirp signals are used in the first transmission frame and the second transmission frame, the distance ranges that can be detected in the first transmission frame and the second transmission frame may be set to be different from each other. In this case, for example, the frequency sweep change rate may be set to be the same (Dₘ₁=Dₘ₂), and the range gates in the first transmission frame and the second transmission frame may be set to different values (T_{sw1}≠T_{sw2}).

For example, by setting the distance range that can be detected in the first transmission frame to be wider than the distance range that can be detected in the second transmission frame, radar apparatus 1 can detect the position information of a walking target approaching the detection area earlier, and can perform the position following at an earlier stage. In this case, for example, chirp signals such as follows may be used: the range of distance information represented by the distance index of a chirp signal transmitted in the first transmission frame is wider than the range of distance information represented by the distance index of a chirp signal transmitted in the second transmission frame. For example, the range gates in the first transmission frame and the second transmission frame may be set to different values (for example, a setting in which Dₘ₁=Dₘ₂ and T_{sw1}>T_{sw2}).

In addition, when different chirp signals are used in the first transmission frame and the second transmission frame, modulation parameters may be set in such a way that the distance information represented by the distance index is partially the same between the first transmission frame and the second transmission frame. For example, modulation parameters may be set in such a way that the frequency sweep change rate of one chirp signal is an integer multiple of the frequency sweep change rate of the other chirp signal. As a result, when the biological detection processing including a chirp signal included in the first transmission frame is performed in addition to the second transmission frame, radar apparatus 1 can reduce the influence of the phase variation due to the difference in the distance information, and can suppress the deterioration influence on the biological detection performance.

Beat frequency analyzer 208 outputs a frequency spectrum in which a peak appears at a beat frequency corresponding to the delay time of a reflected wave signal (radar reflected wave) by the FFT processing. In the FFT processing, for example, beat frequency analyzer 208 may perform multiplication by a window function coefficient such as of the Han window or the Hamming window. The use of a window function coefficient can suppress sidelobes around the beat frequency peak.

Note that, when N_{data1} or N_{data2} is not a power of 2, for example, by including zero-padded data, the FFT processing can be performed with a data size of a power of 2. In this case, the number of data including the zero-padded data may be regarded as N_{data1} or N_{data2}, and the same treatment may be performed regardless of whether N_{data1} or N_{data2} is a power of 2.

Here, in signal processor 206 of z-th antenna system processor 201, a beat frequency response output from beat frequency analyzer 208 obtained by transmitting the m_{b}-th chirp signal in the s_{b}-th first transmission frame is represented by “RFT_{z,sb}(f_{b1}, m_{b})”. Here, f_{b1} represents a beat frequency index and corresponds to an index number (bin number) of the FFT. For example, f_{b1}=0 to N_{data1}/2−1, z=an integer of 1 to Na, m_{b}=an integer of 1 to N_{CB}, and s_{b}=an integer of 1 to N_{FB}. A smaller the beat frequency index f_{b1} indicates a beat frequency with a smaller delay time of a reflected wave signal (for example, the distance to a target object is small).

Similarly, in signal processor 206 of z-th antenna system processor 201, a beat frequency response output from beat frequency analyzer 208 obtained by transmitting the mᵥ-th chirp signal in the sᵥ-th second transmission frame is represented by “RFT_{z,sv}(f_{b2}, mᵥ)”. Here, f_{b2} represents a beat frequency index and corresponds to an index number (bin number) of the FFT. For example, f_{b2}=0 to N_{data2}/2−1, z=an integer of 1 to Na, mᵥ=an integer of 1 to N_{CV}, and sᵥ=an integer of 1 to N_{FVALL}.

Here, N_{FVAll} may be, for example, the number of second transmission frames with the unit of biological detection processing. Here, N_{FVAll}≥N_{fv}. In addition, N_{FB} is, for example, the number of first transmission frames in the number of second transmission frames with the unit of biological detection processing. For example, N_{FB} may be set to N_{FVAll}/N_{fv}.

In addition, the beat frequency indices f_{b1} and f_{b2} may be converted into the distance information R(f_{b1}) and R(f_{b2}) using, for example, Expression (12) and Expression (13). Hereinafter, the beat frequency index is also referred to as a “distance index”.

$$\begin{matrix}( Expression 12 ) & \\ R ( {f}_{{b}_{1}} ) = \frac{{c}_{0}}{2 {B}_{{w}_{1}}} {f}_{{b}_{1}} & [ 12 ]\end{matrix}$$

$$\begin{matrix}( Expression 13 ) & \\ R ( {f}_{{b}_{2}} ) = \frac{{c}_{0}}{2 {B}_{{w}_{2}}} {f}_{{b}_{2}} & [ 13 ]\end{matrix}$$

Here, B_{w1} and B_{w2} each represent a frequency sweep bandwidth in the range gate in a chirp signal of each of the first transmission frame and the second transmission frame, and C₀ represents the speed of light.

Output switch 209 switches the output destination of beat frequency analyzer 208 to either storage 210 or Doppler analyzer 211 based on, for example, an output control signal from transmission controller 105. Here, the output control signal may indicate that the output destination of beat frequency analyzer 208 is Doppler analyzer 211 in the case of transmission in the first transmission frame and that the output destination of beat frequency analyzer 208 is storage 210 in the case of transmission in the second transmission frame.

Storage 210 stores the signal input from output switch 209. For example, storage 210 may store the reception signal for each of the Nt transmission antennas based on the beat frequency response RFT_{z,sb}(f_{b1}, m_{b}) output from beat frequency analyzer 208 in the case of transmission in the second transmission frame. Here, f_{b2}=0 to N_{data2}/2−1, z=1 to Na, mᵥ=1 to N_{CV}, and sᵥ=1 to N_{FVALL}.

For example, in the mᵥ-th transmission period T_{CV} of the second transmission frame, when the chirp signal is output from the mod(mᵥ−1, Nt)+1-th transmission antenna using the time division multiplexing transmission, the beat frequency response from the n-th transmission antenna (Tx #n) among the beat frequency responses RFT_{z,sv}(f_{b2}, mᵥ) output from beat frequency analyzer 208 is a beat frequency response satisfying mod(mᵥ−1, Nt)+1=n. Storage 210 sequentially stores the beat frequency response from the n-th transmission antenna in a reception response storage block of the n-th transmission antenna. Hereinafter, the beat frequency response RFT_{z,sv}(f_{b2}, mᵥ) in the n-th transmission antenna to be stored in storage 210 is represented by “MRFT_{z,n}(f_{b2}, mₜ)”.

Here, mₜ is a storage index assigned in the order of storage in the reception response storage block of the n-th transmission antenna, and mₜ=1 to N_{TPA}×N_{FVAll}.

In FIG. 2, Doppler analyzer 211 performs, via output switch 209, the Doppler analysis for each distance index f_{b1} using the beat frequency response RFT_{z,sb}(f_{b1}, m_{b}) of the first transmission frame output from beat frequency analyzer 208 during the transmission in the first transmission frame.

Hereinafter, an operation example of Doppler analyzer 211 when the Doppler multiplexing (DDM) or the code multiplexing (CDM) is used during the transmission in the first transmission frame will be described.

### (1) Operation Example of Doppler Analyzer 211 when Doppler Multiplexing (DDM) is Used

For example, when the number N_{CB} of chirp signals transmitted in the first transmission frame is a power of 2, the FFT processing can be applied to the Doppler analysis of Doppler analyzer 211. In this case, the FFT size is N_{CB}, and the maximum Doppler frequency that is derived from the sampling theorem and in which no aliasing occurs is ±1/(2×T_{CB}). In addition, the Doppler frequency interval of the Doppler frequency index fs is 1/(N_{CB}×T_{CB}), and the range of the Doppler frequency index fs is: fs=−N_{CB}/2, . . . , 0, . . . , N_{CB}/2−1. Hereinafter, as an example, the case where N_{CB} is a power of 2 will be described. Note that, when N_{CB} is not a power of 2, for example, zero-padded data can be included to perform FFT processing with the number of data sizes (FFT sizes) equal to a power of 2. In addition, Doppler analyzer 211 may perform multiplication by a window function coefficient such as of the Han window or the Hamming window. The use of a window function coefficient can suppress sidelobes around the beat frequency peak.

For example, the output “VFT_{z,sb}(f_{b1}, fₛ)” of Doppler analyzer 211 of z-th signal processor 206 in the s_{b}-th first transmission frame is shown in Expression (14). Note that j is an imaginary unit, and z=1 to Na and s_{b}=1 to N_{FB}.

$$\begin{matrix}( Expression 14 ) & \\ {VFT}_{z , {s}_{b}} ( {f}_{{b}_{1}} , {f}_{s} ) = {\sum }_{{m}_{b} = 0}^{{N}_{CB} - 1} {RFT}_{z , {s}_{b}} ( {f}_{{b}_{1}} , {m}_{b} ) exp [ - j \frac{2 π {m}_{b} {f}_{s}}{{N}_{CB}} ] & [ 14 ]\end{matrix}$$

Here, N_{FVAll} may be, for example, the number of second transmission frames with the unit of biological detection processing. Here, N_{FVAll}≥N_{fv} is set.

In addition, N_{FB} is, for example, the number of first transmission frames in the number of second transmission frames with the unit of biological detection processing. For example, N_{FB} may be set to ceil(N_{FVAll}/N_{fv}).

### (2) Operation Example of Doppler Analyzer 211 when Code Multiplexing (CDM) is Used

For example, corresponding to the orthogonal codes Code_{ncm}={OC_{ncm}(1), OC_{ncm}(2), . . . , OC_{ncm}(Loc)}, Doppler analyzer 211 includes a plurality of Doppler analyzers 211 corresponding to the code length Loc, which is used for code multiplexing.

For example, when the noc-th code element OC_{ncm}(noc) is transmitted, the beat frequency response of the reception signal therefor is input to the noc-th Doppler analyzer 211. For example, a beat frequency response is input to each Doppler analyzer 211 for each transmission period (Loc×T_{CB}). Then, the noc-th Doppler analyzer 211 performs the Doppler analysis for each distance index f_{b1} using the beat frequency response of N_{CB}/Loc (=Ncode) transmission periods among the N_{CB} transmission periods. Here, noc is an index of the code element, and noc=1 to Loc.

For example, when Ncode is a power of 2, the FFT processing can be applied to the Doppler analysis of Doppler analyzer 211. In this case, the FFT size is Ncode, and the maximum Doppler frequency that is derived from the sampling theorem and in which no aliasing occurs is ±1/(2Loc×T_{CB}). In addition, the Doppler frequency interval of the Doppler frequency index fs is 1/(Ncode×Loc×T_{CB}), and the range of the Doppler frequency index fs is −Ncode/2, . . . , 0, . . . , Ncode/2−1.

The following description will be given of the case where Ncode is a power of 2, as an example. When Ncode is not a power of 2, for example, zero-padded data can be included to perform FFT processing with the number of data sizes (FFT sizes) equal to a power of 2. In the FFT processing, Doppler analyzer 211 may perform multiplication by a window function coefficient such as of the Han window or the Hamming window. The application of a window function can suppress sidelobes around the beat frequency peak.

For example, the output VFT_{z,sb,noc}(f_{b1}, fₛ) of the n_{oc}-th Doppler analyzer 211 in z-th signal processor 206 is shown in Expression (15). Note that j is an imaginary unit, and z=1 to Na.

$$\begin{matrix}( Expression 15 ) & \\ {VFT}_{z , {s}_{b} , noc} ( {f}_{{b}_{1}} , {f}_{s} ) = {\sum }_{{m}_{b} = 0}^{{N}_{code} - 1} {RFT}_{z , {s}_{b}} ( {f}_{{b}_{1}} , {L}_{oc} \times s + noc ) ) exp [ - j \frac{2 π {m}_{b} {f}_{s}}{{N}_{code}} ] & [ 15 ]\end{matrix}$$

The foregoing description has been given of the processing performed by the components of signal processor 206.

In FIG. 2, CFAR section 212 may perform the CFAR processing (for example, selective extraction of the local peak of a radar reflected wave (reception signal) by an adaptive threshold value determination) using the output of Doppler analyzer 211 in each of signal processors 206 of the first to Na-th antenna system processors 201. CFAR section 212 extracts a distance index (hereinafter, represented by “f_{b1_cf}”) providing a peak signal and a Doppler frequency index (hereinafter, represented by “f_{s_cf}”) and outputs the extracted indices to multiplex separator 213 and signal extractor 216.

CFAR section 212 may perform, for example, the following CFAR processing for each first transmission frame.

For example, CFAR section 212 may perform the power addition on the outputs (for example, VFT_{z,sb}(f_{b1}, fₛ) or VFT_{z,sb,noc}(f_{b1}, fₛ)) of Doppler analyzers 211 in signal processors 206 of the first to Na-th antenna system processors 201 in the s_{b}-th first transmission frame, and perform two-dimensional CFAR processing with the distance axis and the Doppler frequency axis (for example, corresponding to the relative speed) or CFAR processing that combines one-dimensional CFAR processing to another. Here, s_{b}=1 to N_{FB}.

Note that, for example, the processing disclosed in NPL 5 may be applied to the operation of the CFAR processing.

### (1) Operation Example of CFAR Section 212 when Doppler Multiplexing (DDM) is Used in the Case of Transmission in First Transmission Frame

Here, when, for example, Expression (6) is used as the phase rotation amount φ_{ndm} for imparting DOP_{ndm} during the Doppler multiplexing transmission, the intervals between the Doppler shift amounts in the Doppler frequency domain in the output of Doppler analyzer 211 are even intervals. When the interval ΔFD between the Doppler shift amounts is expressed in terms of Doppler frequency intervals, ΔFD=N_{CB}/N_{DM}. Therefore, in the output of Doppler analyzer 211, in the Doppler frequency domain, the peaks of the Doppler multiplexed signals are detected at the interval of ΔFD.

Therefore, CFAR section 212 may, for example, divide each output of Doppler analyzer 211 in a range of ΔFD, perform power addition (for example, referred to as “Doppler domain compression”) on the signal peak positions for the respective divided ranges as shown in Expression (16), and then perform the CFAR processing (for example, referred to as “Doppler domain compression CFAR processing” and expressed as DC-CFAR). Here, f_{sc}=−ΔFD/2, . . . , (ΔFD/2)−1. For example, in the case of ΔFD=N_{CB}/N_{DM}, f_{sc}=−N_{CB}/(2N_{DM}), . . . , (N_{CB}/(2N_{DM}))−1. Note that the DC-CFAR is described in, for example, PTL 2, and the detailed description thereof will be omitted.

$$\begin{matrix}( Expression 16 ) & \\ {PowerFT}_{{s}_{b}} ( {f}_{b} , {f}_{sc} ) = {\sum }_{ndm = 1}^{{N}_{DM}} {\sum }_{z = 1}^{{N}_{a}} {\vert {VFT}_{z , {s}_{b}} ( {f}_{{b}_{1}} , {f}_{sc} + ( ndm - 1 ) \times Δ FD ) \vert}^{2} & [ 16 ]\end{matrix}$$

CFAR section 212 using the DC-CFAR adaptively sets a threshold value, and outputs, to multiplex separator 213, f_{b1_cf} and f_{sc_cf} providing reception power greater than the threshold value, and the reception power information Power FT(f_{b1_cf}, f_{sc_cf}+(ndm−1)×ΔFD) in the Doppler frequency index (f_{sc_cf}+(ndm−1)×ΔFD) of the N_{DM} Doppler multiplexed signals and the output VFT_{z,sb}(f_{b1_cf}, f_{sc_cf}+(ndm−1)×ΔFD) of Doppler analyzer 211. Here, ndm=1 to N_{DM}. In addition, CFAR section 212 adaptively sets a threshold value, and outputs f_{b1_cf} and f_{sc_cf} providing reception power greater than the threshold value to signal extractor 216.

### (2) Operation Example of CFAR Section 212 when Code Multiplexing (CDM) is Used in the Case of Transmission in First Transmission Frame

CFAR section 212 may perform the power addition on the outputs VFT_{z,sb,noc}(f_{b1}, fₛ) of the Loc Doppler analyzers 211 in the signal processors 206 of the first to the Na-th in the s_{b}-th first transmission frame as shown in Expression (17), and then perform the CFAR processing. The CFAR processing is described in, for example, NPL 5, and the operation description thereof will be omitted.

$$\begin{matrix}( Expression 17 ) & \\ {PowerFT}_{{s}_{b}} ( {f}_{b 1} , {f}_{s} ) = {\sum }_{noc = 1}^{{L}_{oc}} {\sum }_{z = 1}^{{N}_{a}} {\vert {VFT}_{z , {s}_{b} , noc} ( {f}_{b 1} , {f}_{s} ) \vert}^{2} & [ 17 ]\end{matrix}$$

Next, an operation example of multiplex separator 213 illustrated in FIG. 2 will be described.

Hereinafter, an operation example of multiplex separator 213 when the Doppler multiplexing (DDM) is used during the transmission in the first transmission frame will be described.

Note that the operation example of multiplex separator 213 when the code multiplexing (CDM) is used during the transmission in the first transmission frame is described in, for example, PTL 3, and the operation description thereof will be omitted. In addition, the operation after the demultiplexing is the same as the operation when the Doppler multiplexing (DDM) is used.

In addition, hereinafter, an example of the processing of the Doppler separation when the DC-CFAR is used in CFAR section 212 will be described.

For example, multiplex separator 213 separates the Nt Doppler-multiplexed and transmitted signals using reception power information—that is in the Doppler frequency indices of the N_{DM} Doppler multiplexed signals in f_{b1_cf} and f_{sc_cf} input from CFAR section 212 in the s_{b}-th first transmission frame—and the outputs of the Doppler analyzers 211 of signal processors 206 of the first to the Na-th, discriminates (for example, determines or identifies) the transmission antennas, and outputs the discrimination result to direction estimator 214. Here, s_{b}=1 to N_{FB}. In addition, multiplex separator 213 outputs, for example, the Doppler frequency (for example, the Doppler speed or the relative speed) based on the discrimination result of the transmission antenna.

Note that the separation processing of Doppler multiplexed signals is technically disclosed in, for example, PTL 2 and PTL 4, and the detailed description thereof will be omitted.

For example, the separation processing of Doppler multiplexed signals when the even interval Doppler shift amount as shown in Expression (6) is set during the Doppler multiplexing transmission is technically disclosed in PTL 4, and the Doppler detection (relative speed detection) of a target object becomes possible in the Doppler frequency range of ±1/(2×N_{DM}×T_{CB}).

For example, the separation processing of Doppler multiplexed signals when the uneven interval Doppler shift amount as shown in Expression (7) is set in the Doppler multiplexing transmission is technically disclosed in PTL 2, and the Doppler detection (relative speed detection) of a target object becomes possible in the Doppler frequency range of ±1/(2×T_{CB}).

In FIG. 2, direction estimator 214 performs the direction estimation processing of a target object using, for example, a reception signal for each transmission antenna obtained by separating transmission multiplexed signals in multiplex separator 213 at the distance index and the Doppler frequency indices extracted in CFAR section 212, in the s_{b}-th first transmission frame. Then, direction estimator 214 outputs, to position follower 215, the distance information and the Doppler frequency (for example, the Doppler speed or the relative speed) information of the target object and the direction estimation result as the position information of the target object based on the distance index and the Doppler frequency index extracted in CFAR section 212 in the s_{b}-th first transmission frame. Here, s_{b}=1 to N_{FB}.

For example, direction estimator 214 generates the virtual reception array correlation vector h(f_{b_cf}, f_{sc_cf}) as shown in Expression (18) and performs the direction estimation processing.

$$\begin{matrix}( Expression 18 ) & \\ h ( {f}_{b 1 \_ cf} , {f}_{sc \_ cf} ) = [ \begin{matrix}{C}_{1 , 1} \times {y}_{1 , 1} ( {f}_{b 1 \_ cf} , {f}_{sc \_ cf} ) \\ {C}_{1 , 2} \times {y}_{2 , 1} ( {f}_{b 1 \_ cf} , {f}_{sc \_ cf} ) \\ ⋮ \\ {C}_{Na , 1} \times {y}_{Na , 1} ( {f}_{b 1 \_ cf} , {f}_{sc \_ cf} ) \\ {C}_{1 , 2} \times {y}_{1 , 2} ( {f}_{b 1 \_ cf} , {f}_{sc \_ cf} ) \\ {C}_{2 , 2} \times {y}_{2 , 2} ( {f}_{b 1 \_ cf} , {f}_{sc \_ cf} ) \\ ⋮ \\ {C}_{Na , 2} \times {y}_{Na , 2} ( {f}_{b 1 \_ cf} , {f}_{sc \_ cf} ) \\ ⋮ \\ {C}_{1 , Nt} \times {y}_{1 , Nt} ( {f}_{b 1 \_ cf} , {f}_{sc \_ cf} ) \\ {C}_{1 , Nt} \times {y}_{2 , Nt} ( {f}_{b 1 \_ cf} , {f}_{sc \_ cf} ) \\ ⋮ \\ {C}_{Na , Nt} \times {y}_{Na , Nt} ( {f}_{b 1 \_ cf} , {f}_{sc \_ cf} )\end{matrix} ] & [ 18 ]\end{matrix}$$

In Expression (18), y_{z,n}(f_{b1_cf}, f_{sc_cf}) represents a reception signal in the z-th reception antenna of the n-th transmission antenna separated by multiplex separator 213, and is associated using the output VFT_{z,sb}(f_{b1_cf}, f_{sc_cf}+(ndm−1)×ΔFD) of Doppler analyzer 211.

Here, the virtual reception array correlation vector h(f_{b_cf}, f_{s_cf}) is, for example, a vector including elements of (Nt×Na), namely the product of the number of transmission antennas Nt and the number of reception antennas Na, as shown in Expression (18). In addition, C_{z,n} is an array correction value for correcting a phase deviation and an amplitude deviation between the n-th transmission antenna and the z-th reception antenna.

Direction estimator 214 calculates the spatial profile by, for example, varying the azimuth direction θᵤ in the direction estimation evaluation function value P_{H}(θᵤ) within a predetermined angle range. Direction estimator 214 may extract a predetermined number of maximal peaks in the calculated spatial profile in descending order of magnitude, and output the azimuth direction of the maximal peak to position follower 215 as an arrival direction estimation value (for example, a position measurement output).

Note that there are various methods such as a beam forming method, Capon, and MUSIC for the direction estimation evaluation function value P_{H}(θᵤ) by the arrival direction estimation algorithm. For example, the estimation method disclosed in NPL 6 may be used.

For example, the beam forming method can be represented by Expression (19). Note that, in Expression (19), the superscript H is a Hermitian transpose operator.

(Expression 19)

P_{H}(θᵤ)=a^{H}(θᵤ)h(f_{b1_cf},s_{c_cf}) [19]

In addition, in Expression (19), a(θᵤ) indicates a direction vector of the virtual reception array for an incident wave at the azimuth direction θᵤ at the center frequency fc of the radar transmission signal, and is represented by Expression (20) when the Nt×Na virtual reception arrays are linearly arranged at even intervals d_{H}. In Expression (20), λ is a wavelength of the radar transmission signal (for example, the chirp signal) in the case of the center frequency f_{c}, and is λ=C₀/f_{c}.

$$\begin{matrix}( Expression 20 ) & \\ a ( {θ}_{u} ) = [ \begin{matrix}1 \\ exp ( - j 2 π {d}_{H} sin {θ}_{u} / λ ) \\ ⋮ \\ exp ( - j 2 π ( {N}_{t} {N}_{a} - 1 ) {d}_{H} sin {θ}_{u} / λ )\end{matrix} ] & [ 20 ]\end{matrix}$$

In addition, the azimuth direction θᵤ is a vector that changes at a predetermined azimuth interval β₁ within the azimuth range in which the arrival direction estimation is performed. For example, θᵤ is set as follows.

θᵤ=θmin+uβ₁, integer u=0 to NU

NU=floor[(θmax−θmin)/β₁]+1

Here, floor(x) is a function that returns the largest integer value not greater than real number x.

Note that, in the above-described example, the example has been described in which direction estimator 214 calculates the azimuth direction as the arrival direction estimation value, but the present disclosure is not limited thereto, and the arrival direction estimation of the elevation direction or, by using MIMO antennas arranged in a rectangular lattice, the arrival direction estimation of the azimuth direction and the elevation direction is also possible. For example, direction estimator 214 may calculate the azimuth direction and the elevation direction as the arrival direction estimation values and use the calculated values as the position measurement output.

In FIG. 2, position follower 215 performs target object following processing (tracking processing) based on the target object position information from direction estimator 214, and outputs the processing result as the position measurement information. In addition, position follower 215 outputs the processing result to beam-weight multiplier 217.

Position follower 215, for example, uses the position information of the target object for each first transmission frame input from direction estimator 214 to estimate whether the target object is the same as a target object detected using a past first transmission frame, and assigns the same identification number (ID number) to the target object estimated to be the same as the past target object information. For example, position follower 215 may perform the following processing in which the tracking filter processing such as the Kalman filter is performed, and output the processing result as the position measurement information. For example, the processing disclosed in NPL 7 can be applied to the target object following processing (tracking processing), and the detailed description thereof will be omitted here.

In FIG. 2, signal extractor 216 extracts the distance index including a living body based on the beat frequency response MRFT_{z,n}(f_{b2}, mₜ) stored in storage 210 during the transmission in the second transmission frame and the information (f_{b1_cf}, f_{s_cf}) of the distance index and the Doppler frequency index extracted by CFAR section 212. Hereinafter, an operation example of signal extractor 216 will be described.

Signal extractor 216 extracts, for example, a portion of the beat frequency response stored in storage 210 (for example, the beat frequency response of the second transmission frame transmitted between the s_{b}-th first transmission frame and the (s_{b}+1)-th first transmission frame, for example, the stored beat frequency response of m_{t_sel,sb}=N_{TPA}×N_{fv}×(s_{b}−1)+1, . . . , N_{TPA}×N_{fv}×s_{b}), based on the information (f_{b1_cf}, f_{s_cf}) of the distance index and the Doppler frequency index, namely the output of CFAR section 212 based on the s_{b}-th first transmission frame. Hereinafter, the beat frequency response extracted by signal extractor 216 and stored in storage 210 is represented by “MRFT_{z,n}(f_{b2_cf}, m_{t_sel,sb})”.

Here, f_{b2_cf} represents a distance index that matches or substantially matches the distance information R(f_{b1}) indicated by the distance index f_{b1_cf} (for example, R(f_{b1_cf})≈R(f_{b2_cf})) or a plurality of distance indices that are within a predetermined distance range including the distance information R(f_{b1}). For example, R(f_{b1_cf})−Ra≤R(f_{b2_cf})≤R(f_{b1_cf})+Ra, and Ra represents a predetermined distance. Here, z=1 to Na, n=1 to Nt, and s_{b}=1 to N_{FB}.

Here, when radar apparatus 1 is fixedly installed indoors or outdoors or is fixedly installed in a vehicle and detects a target object in the vehicle, in the extraction operation of signal extractor 216, an extracted target object whose Doppler frequency index information f_{s_cf} (namely the output of CFAR section 212) is zero (or near zero) can be regarded as the detection of a stationary object including a living body. When a living body is in a stationary state, the accuracy of the biological detection can be relatively high. The output of signal extractor 216, which can be regarded as such a stationary object, is also represented by “StaticMRFT_{z,n}(f_{b2_cf}, m_{t_sel,sb})”.

In addition, in the extraction operation of signal extractor 216, an extracted target object whose Doppler frequency index information f_{s_cf} (namely the output of CFAR section 212) is not zero (or not near zero) can be regarded as the detection of a moving target object including a living body. When a living body is in a moving state, the biological detection includes the walking or the body movement, and therefore, in the biological detection, the improvement in the biological detection accuracy can be achieved by adding the correction of reducing the Doppler frequency due to such walking or body movement. The output of signal extractor 216, which can be regarded as such a moving target object, is also represented by “MovingMRFT_{z,n}(f_{b2_cf}, m_{t_sel,sb})”.

In addition, when the Doppler frequency index information f_{s_cf} (namely the output of CFAR section 212) can be regarded as the moving target object as described above, signal extractor 216 may multiply the Doppler phase variation correction coefficient that cancels the phase variation due to the Doppler frequency due to the walking or the body movement, and output the result as the output of signal extractor 216. For example, signal extractor 216 may output, as MovingMRFT_{z,n}(f_{b2_cf}, m_{t_sel,sb}), a result of multiplying the output MRFT_{z,n}(f_{b2_cf}, m_{t_sel,sb}) of storage 210 by a Doppler phase variation correction coefficient as shown in Expression (21).

$$\begin{matrix}( Expression 21 ) & \\ {MovingMRFT}_{z , n} ( {f}_{b 2 \_ cf} , {m}_{t \_ sel , {s}_{b}} ) = {MRFT}_{z , n} ( {f}_{b 2 \_ cf} , {m}_{t \_ sel , {s}_{b}} ) exp [ - j \frac{2 π ( {m}_{{t}_{sel , {s}_{b}}} - {N}_{TPA} \times {N}_{fv} \times ( {s}_{b} - 1 ) - 1 ) {f}_{s \_ cf}}{{N}_{CB}} \times \frac{{T}_{VM}}{{T}_{CB}} ] & [ 21 ]\end{matrix}$$

Alternatively, signal extractor 216 may extract the distance index including a living body based on the beat frequency response MRFT_{z,n}(f_{b2}, mₜ) stored in storage 210 during the transmission in the second transmission frame. In the case of distance index including the living body, for example, a position change of the body surface such as breathing or a heartbeat occurs. The position change of the breathing component is larger than that of the heartbeat component, and is easily detected as the position change. Therefore, by setting the number N_{FVAll} of second transmission frames with the unit of biological detection processing in consideration of the observation time including at least one cycle of the position change due to the breathing, the biological detection is facilitated. Hereinafter, an operation example thereof will be described.

For example, signal extractor 216 calculates the biological detection evaluation index for each distance index using the beat frequency response MRFT_{z,n}(f_{b2}, mₜ) stored in storage 210 during the transmission in the second transmission frame. As the biological detection evaluation index, for example, the standard deviation or the variance of the beat frequency response for each distance index may be used. Alternatively, as the biological detection evaluation index, for example, the standard deviation or the variance value of the time difference value (for example, MRFT_{z,n}(f_{b2}, mₜ)−MRFT_{z,n}(f_{b2}, mₜ−1)) of the beat frequency response for each distance index may be used. For example, when the variance of the beat frequency response for each distance index is used as the biological detection evaluation index, the biological detection evaluation index is represented by Expression (22). Here, VDM_{z,n}(f_{b2}) represents a biological detection evaluation index value in the beat frequency response of the distance index f_{b2} transmitted from the n-th transmission antenna and received by the z-th reception antenna.

$$\begin{matrix}( Expression 22 ) & \\ {VDM}_{z , n} ( {f}_{b 2} ) = {\sum }_{{m}_{t} = 1}^{{N}_{TPA} \times {N}_{FV ALL}} {\{ {MRFT}_{z , n} ( {f}_{b 2} , {m}_{t} ) - \frac{{\sum }_{{m}_{t} = 1}^{{N}_{TPA} \times {N}_{FV ALL}} {MRFT}_{z , n} ( {f}_{b 2} , {m}_{t} )}{{N}_{TPA} \times {N}_{FV ALL}} \}}^{2} & [ 22 ]\end{matrix}$$

Note that signal extractor 216 may use the biological detection evaluation index value obtained by summing the plurality of transmission and reception antennas as in Expression (23). As a result, the noise influence can be reduced, and the biological detection sensitivity can be improved.

$$\begin{matrix}( Expression 23 ) & \\ VDM ( {f}_{b 2} ) = {\sum }_{z = 1}^{{N}_{a}} {\sum }_{z = 1}^{{N}_{t}} {VDM}_{z , n} ( {f}_{b 2} ) & [ 23 ]\end{matrix}$$

Signal extractor 216 extracts a local peak that satisfy the condition in which the biological detection evaluation index value is equal to or higher than a predetermined value or the local peak and the distance index around the local peak (hereinafter, the f_{b2} index satisfying the above-described extraction condition is represented by “f_{b2_VDM}”) and the beat frequency response MRFT_{z,n}(f_{b2_VDM}, mₜ) of the storage index mₜ (index indicating the serial number of the beat frequency response of the plurality of second transmission frames). The output of signal extractor 216 that can be regarded as the biological target object using such a biological detection evaluation index is also represented by “VdmMRFT_{z,n}(f_{b2_VDM}, mₜ)”. Here, mₜ=1 to N_{TPA}×N_{FVAll}.

Note that signal extractor 216 may output the three types of extraction results described above, or may extract at least one of the three types of extraction results. For example, signal extractor 216 may extract StaticMRFT_{z,n}(f_{b2_cf}, m_{t_sel,sb}) that can be regarded as a stationary object allowing relatively accurate biological detection but not the others. Alternatively, signal extractor 216 may extract, for example, two types, namely StaticMRFT_{z,n}(f_{b2_cf}, m_{t_sel,sb}) that can be regarded as a stationary object allowing relatively accurate biological detection and VdmMRFT_{z,n}(f_{b2_VDM}, mₜ) using the biological detection evaluation index value.

In addition, when the number of chirp signal transmissions N_{TPA} for each transmission antenna in the second transmission frame is 2 or more, signal extractor 216 may perform the above-described extraction processing by using a beat frequency response SumMRFT_{z,n}(f_{b2}, mₜₛ) instead of MRFT_{z,n}(f_{b2_VDM}, mₜ) and using mₜₛ instead of mₜ. The beat frequency response SumMRFT_{z,n}(f_{b2}, mₜₛ) is a result of the below in-phase addition processing (referred to as in-phase addition in which the complex number value addition processing is performed). Here, mₜₛ represents the index number of a second transmission frame after the in-phase addition (assigned a serial number at the unit of biological processing), and mₜₛ=1 to N_{FVAll}.

In the case of performing the in-phase addition, signal extractor 216 may perform the in-phase addition (referred to as in-phase addition in which the complex number value addition processing is performed) as in Expression (24) based on the beat frequency response MRFT_{z,n}(f_{b2_VDM}, mₜ) stored in storage 210 for the plurality of times (N_{TPA}≥2) of the chirp signal for each transmission antenna in the second transmission frame.

$$\begin{matrix}( Expression 24 ) & \\ {SumMRFT}_{z , n} ( {f}_{b 2} , {m}_{ts} ) = {\sum }_{s = 1}^{{N}_{TPA}} {MRFT}_{z , n} ( {f}_{b 2} , {N}_{TPA} \times ( {m}_{ts} - 1 ) + s ) & [ 24 ]\end{matrix}$$

With such in-phase addition processing, the transmission time difference between the chirp signals for each of the plurality of N_{TPA} transmission antennas is set to T_{CV}×Nt, which is set to a time sufficiently shorter than the reception phase change in the biological detection (breathing or heartbeat). As long as there is no abrupt change in the body movement such as the walking or the body movement, the reception phase difference can achieve an SNR improvement effect by the in-phase (coherent) addition gain.

On the other hand, the phase change “DP_{z,n}(f_{b2_VDM}, mₜ)” between the beat frequency responses of the chirp signals for each of the plurality of N_{TPA} transmission antennas transmitted in the second transmission frame is referred to as “phase variation in second transmission frame”. The phase variation in the second transmission frame at the transmission time difference T_{CV}×Nt can be represented by, for example, Expression (25). Here, angle[x] is a function that outputs an argument of a complex number x.

(Expression 25)

DP_{z,n}(f_{b2_VDM},mₜ)=angle[MRFT_{z,n}(f_{b2_VDM},mₜ)−MRFT_{z,n}(f_{b2_VDM},mₜ₋₁)] [25]

When the phase variation in the second frame is larger than a predetermined value, the walking or the body movement change is assumed to occur and can be used for the walking/body movement detection, and thus signal extractor 216 may output the extraction result to subsequent beam-weight multiplier 217 and vital detector 218. For example, when the phase variation in the second frame is larger than the predetermined value, the reliability of the biological detection is more likely to become low, and thus, for example, vital detector 218 may add the following processing as a biological detection reliability evaluation value: a time section of the second frame in which the phase variation in mₜ second frame becomes larger than the predetermined value is regarded as a time section in which the reliability of the biological detection is low and the process of excluding or estimating a highly reliable time section is used as an alternative output.

When the output from signal extractor 216 includes the reception signal by the plurality of transmission antennas or the plurality of reception antennas, beam-weight multiplier 217 multiplies the output by the beam weight that improves the reception SNR and outputs the result to vital detector 218. With such an operation of beam-weight multiplier 217, the heartbeat detection accuracy in the MIMO radar using the plurality of transmission and reception antennas is improved.

Note that signal extractor 216 can output the three types of extraction results (StaticMRFT_{z,n}(f_{b2_cf}, m_{t_sel,sb}), MovingMRFT_{z,n}(f_{b2_cf}, m_{t_sel,sb}), and VdmMRFT_{z,n}(f_{b2_VDM}, mₜ)), but the processing of beam-weight multiplier 217 is the same for any of the three types. Therefore, an output from signal extractor 216 will be represented by “Y_{z,n}(f_{b2sel}, mₜₛₑₗ)”, and an operation example of beam-weight multiplier 217 will be described. Here, z=1 to Na and n=1 to Nt, f_{b2sel} indicates the extracted distance index, and mₜₛₑₗ indicates the index number (serial number at the unit of biological processing) of the extracted second transmission frame.

Beam-weight multiplier 217 may constitute a virtual reception array correlation vector q(f_{b2sel}, mₜₛₑₗ) having Nt×Na elements as in Expression (27) based on the output Y_{z,n}(f_{b2sel}, mₜₛₑₗ) from signal extractor 216, and may calculate a virtual reception array correlation matrix Q(f_{b2sel}, mₜₛₑₗ) as in Expression (26). In Expression (26), the sum of the cross-correlation matrices based on the virtual reception array correlation vector q(f_{b2sel}, mₜₛₑₗ) shown in Expression (27) is calculated in a range of the index number m^{tsel} of the second transmission frame extracted in signal extractor 216.

$$\begin{matrix}( Expression 26 ) & \\ Q ( {f}_{b 2 sel} ) = {Σ}_{{m}_{tsel}} q ( {f}_{b 2 sel} , {m}_{tsel} ) {q ( {f}_{b 2 sel} , {m}_{tsel} )}^{H} & [ 26 ]\end{matrix}$$

$$\begin{matrix}( Expression 27 ) & \\ q ( {f}_{b 2 sel} , {m}_{tsel} ) = [ \begin{matrix}{Y}_{1 , 1} ( {f}_{b 2 sel} , {m}_{tsel} ) \\ {Y}_{2 , 1} ( {f}_{b 2 sel} , {m}_{tsel} ) \\ ⋮ \\ {Y}_{Na , 1} ( {f}_{b 2 sel} , {m}_{tsel} ) \\ {Y}_{1 , 2} ( {f}_{b 2 sel} , {m}_{tsel} ) \\ {Y}_{2 , 2} ( {f}_{b 2 sel} , {m}_{tsel} ) \\ ⋮ \\ {Y}_{Na , 2} ( {f}_{b 2 sel} , {m}_{tsel} ) \\ ⋮ \\ {Y}_{1 , Nt} ( {f}_{b 2 sel} , {m}_{tsel} ) \\ {Y}_{2 , Nt} ( {f}_{b 2 sel} , {m}_{tsel} ) \\ ⋮ \\ {Y}_{Na , Nt} ( {f}_{b 2 sel} , {m}_{tsel} )\end{matrix} ] & [ 27 ]\end{matrix}$$

Beam-weight multiplier 217 generates the beam weight based on the calculated virtual reception array correlation matrix Q(f_{b2sel}, mₜₛₑₗ). For example, beam-weight multiplier 217 may perform eigenvalue decomposition on the virtual reception array correlation matrix Q(f_{b2sel}, mₜₛₑₗ) and use an eigenvector beam EigVec that provides the eigenvalue as the beam weight. Here, as the eigenvalue is larger, the reception SNR of the reception signal after the beam weight multiplication can be improved. Therefore, beam-weight multiplier 217 may set, for example, the eigenvector of the maximum eigenvalue or the eigenvector of the second largest eigenvalue as the beam weight W(f_{b2sel}).

Beam-weight multiplier 217 outputs the beam reception signal BO(f_{b2sel}, mₜₛₑₗ) calculated as in Expression (28) using the generated beam weight W(f_{b2sel}) to vital detector 218.

(Expression 28)

BO(f_{b2sel},mₜₛₑₗ)=W(f_{b2sel})^{H}q(f_{b2sel},mₜₛₑₗ) [28]

Alternatively, as shown in Expression (29), beam-weight multiplier 217 may set the complex conjugate of the virtual reception array correlation vector q(f_{b2sel}, mₜₛₑₗ) as the beam weight W(f_{b2sel}), thereby improving the reception SNR of the reception signal after the beam weight multiplication. This method does not require eigenvalue decomposition, making it possible to generate beam weights with a low amount of calculation.

(Expression 29)

BO(f_{b2sel},mₜₛₑₗ)=q(f_{b2sel},mₜₛₑₗ)^{H}q(f_{b2sel},mₜₛₑₗ) [29]

Alternatively, beam-weight multiplier 217 may generate the beam weight based on the result of angle measurement included in the position measurement output from position follower 215. For example, when the direction estimation value θ(f_{b_cfar}) of the radar reflected wave at the distance index f_{b_cfar} is the result of angle measurement included in the position measurement output from position follower 215, beam-weight multiplier 217 may generate the beam weight directed to the direction estimation value θ(f_{b_cfar}) of a radar reflected wave as the directivity beam formed by using the virtual reception array correlation vector q(f_{b2sel}, mₜₛₑₗ). In this case, beam-weight multiplier 217 may generate the beam weight by using the direction vector of the virtual reception array and may multiply the beam weight as in Expression (30). Here, the superscript H is a Hermitian transpose operator. In addition, a(θ) indicates the direction vector of the virtual reception array for an incident wave at the azimuth direction θ at the center frequency fc of a transmission signal. As a result, the reception SNR of the reception signal after the beam weight multiplication can be improved. In addition, this method does not require eigenvalue decomposition, making it possible to generate beam weights with a low amount of calculation.

(Expression 30)

BO(f_{b2sel},mₜₛₑₗ)=a(θ(f_{b_cfar}))^{H}q(f_{b2sel},mₜₛₑₗ) [30]

Alternatively, beam-weight multiplier 217 may perform angle measurement processing based on the virtual reception array correlation matrix Q(f_{b2sel}, mₜₛₑₗ) as in Expression (26), and may generate the beam weight based on the result of the angle measurement. The angle measurement processing may be, for example, as follows: calculating the spatial profile by varying the azimuth direction θᵤ in the direction estimation evaluation function value P_{H}(θᵤ) within a predetermined angle range, extracting a predetermined number of maximal peaks of the calculated spatial profile in descending order of magnitude, and using the azimuth direction of the maximal peak as the arrival direction estimation value (for example, the position measurement output). Beam-weight multiplier 217 may include, in the biological detection result output, the biological detection result and the thus estimated arrival direction estimation value and the distance information corresponding to the distance index f_{b2sel} as the position measurement output.

Note that there are various methods such as a beam forming method, Capon, and MUSIC with the arrival direction estimation algorithm for the direction estimation evaluation function value P_{H}(θᵤ). For example, the estimation method disclosed in NPL 6 may be used. For example, when the direction estimation value θ(f_{b2sel}) of a radar reflected wave at the distance index f_{b2sel} is the result of angle measurement by the above-described angle measurement processing, beam-weight multiplier 217 may generate the beam weight directed to the direction estimation value θ(f_{b2sel}) of the radar reflected wave as the directivity beam formed by using the virtual reception array correlation vector q(f_{b2sel}, mₜₛₑₗ). In this case, beam-weight multiplier 217 may generate the beam weight by using the direction vector of the virtual reception array, and may multiply the beam weight of a(θ(f_{b2sel})) instead of a(θ(f_{b_cfar})) in Expression (30).

In FIG. 2, vital detector 218 detects vital information (also referred to as biological information) of a living body such as a human body based on, for example, the output signal from beam-weight multiplier 217. The detection of the vital information may include, for example, breathing detection (for example, breathing number detection) or heartbeat detection (for example, heartbeat number). The methods of the breathing detection and the heartbeat detection are described in, for example, NPL 3, and may be detected by the following processing.

Vital detector 218 may calculate, for example, as in Expression (31), the phase component Ψ(f_{b2sel}, mₜₛₑₗ) of a beam reception signal BO(f_{b2sel}, mₜₛₑₗ) that is the output from beam-weight multiplier 217. Here, f_{b2sel} indicates the distance index extracted by signal extractor 216, m^{tsel} indicates the index number (serial number at the unit of biological processing) of the second transmission frame extracted by signal extractor 216, and includes some or all of the indices from 1 to N_{FVAll}.

(Expression 31)

ψ(f_{b2sel},mₜₛₑₗ)=angle[BO(f_{b2sel},mₜₛₑₗ)] [31]

In addition, when the phase variation is assumed to exceed ±π, vital detector 218 may calculate, for example, the phase component Ψ(f_{b2sel}, mₜₛₑₗ) of the beam reception signal BO(f_{b2sel}, mₜₛₑₗ) as in Expression (32) by using phase unwrapping processing.

(Expression 32)

ψ(f_{b2sel},mₜₛₑₗ)=unwrap[angle[BO(f_{b2sel},mₜₛₑₗ)]] [32]

In addition, the center of the phase change in the complex phase plane may be observed to be shifted from the origin of the complex phase plane due to the influence of a stationary object or the like around a target object. In this case, vital detector 218 may calculate the phase component after adding a correction of setting the center of the phase change to the origin of the complex phase plane (for example, see NPL 3).

In the vital detection processing described above, when the first transmission frame transmission duration is included in addition to the second transmission frame transmission duration as in the case of N_{FVAll}>N_{fv}, vital detector 218 adds processing that takes into account the second transmission frame offset period T_{ofs}.

For example, when the second transmission frame offset period T_{ofs} is set to an integer multiple of T_{VM} (for example, T_{ofs}=N_{ofs}×T_{VM}), the second transmission frame transmission durations before and after T_{ofs} can maintain the consistency of the T_{VM} cycle, and the reception phase change can be sampled at the T_{VM} cycle, which is most suitable. For example, when T_{ofs}=N_{ofs}×T_{VM} is set, the second transmission frame offset period T_{ofs} includes a period in which the N_{ofs} second transmission frames are not transmitted (for example, referred to as a “second transmission frame omission period”). In such a second transmission frame omission period, vital detector 218 may calculate an interpolated value using the reception signals of the second transmission frames before and after the second transmission frame omission period, and may perform the heartbeat detection processing by including the interpolation value in the phase component Ψ(f_{b2sel}, mₜₛₑₗ). With such processing, the deterioration in the accuracy of the heartbeat detection due to the second transmission frame omission period can be reduced. Alternatively, vital detector 218 may calculate the phase component by using a part of transmission signals included in the first transmission frame instead of the interpolation processing (the example thereof will be described in Variation 3). In this case, the deterioration in the accuracy of the heartbeat detection due to the error in the interpolation processing can be suppressed.

In addition, for example, when the second transmission frame offset period T_{ofs} is not set to an integer multiple of T_{VM} (for example, T_{ofs}≠N_{ofs}×T_{VM}), the second transmission frame transmission duration before and after T_{ofs} does not maintain the consistency of the T_{VM} cycle. Therefore, vital detector 218 may perform, for example, resampling processing (interpolation processing of the observation data on the time axis) so that the sampling becomes equivalent to the T_{VM} cycle over the second transmission frame transmission duration before and after T_{ofs}. Alternatively, vital detector 218 may perform the heartbeat detection processing with a detection signal as a unit by using the second transmission frame not including T_{ofs}. In this case, the resampling processing does not need to be performed.

In addition, for example, in order to detect a cycle in which the phase component varies due to changes in the body surface such as breathing or heartbeat (hereinafter, referred to as a “phase component variation cycle”), vital detector 218 may generate signals by removing a noise component or an unnecessary vibration component by allowing the calculated phase component Ψ(f_{b2sel}, mₜₛₑₗ) to individually pass through a filter such as a low pass filter (LPF), a band pass filter (BPF), or a high pass filter (HPF) according to the frequency range corresponding to the expected phase component variation cycle in breathing or heartbeat (for example, referred to as filter processing). As an example, the expected phase component variation frequency range when the breathing number is detected is approximately 0.03 to 0.5 Hz, and the expected phase component variation frequency range when the heartbeat number is detected is approximately 0.67 to 15 Hz. From this, vital detector 218 may apply a filter that allows passing of the frequency range corresponding to each of the vital information (breathing, heartbeat) to be detected. In addition, vital detector 218 may apply, for example, a combination of a plurality of filters.

Alternatively, vital detector 218 may generate, for example, signals by extracting the frequency component corresponding to the expected phase component variation cycle by decomposing the calculated phase component into different frequency components (intrinsic mode functions, IMF) based on a variational mode decomposition (VMD) method or an empirical mode decomposition (EMD) method (this processing is, for example, referred to as mode decomposition processing). In addition, vital detector 218 may generate a signal to which both filter processing and mode decomposition processing are applied.

When the signals obtained by performing the filter processing or the mode decomposition processing include a signal due to the change in the body surface, the periodic change is included. Vital detector 218 can detect the breathing cycle or the heartbeat cycle by observing the variation cycle included in the signals. In addition, vital detector 218 can detect the breathing variability or the heart rate variability (HRV) by observing the change in each cycle. In addition, vital detector 218 can detect the breathing number or the heartbeat number by observing the detected breathing cycle or heartbeat cycle for a predetermined time.

Note that vital detector 218 may perform Fourier frequency analysis or wavelet frequency analysis on the signal obtained by performing the filter processing or the intrinsic mode decomposition processing. For example, vital detector 218 may determine that there is heartbeat information when there is a component exceeding a predetermined peak level (for example, a threshold value) in a predetermined phase component variation frequency range, and may output the phase component variation frequency at the peak as the heartbeat detection result. On the other hand, vital detector 218 may determine that there is no heartbeat information when there is no component exceeding a predetermined peak level (for example, a threshold value) in a predetermined phase component variation frequency range. In addition, when the displacement due to the walking or the body movement is equal to or larger than a predetermined value in the walking/body movement detection using a reception signal in the first transmission frame or the second transmission frame, vital detector 218 may determine that there is no heartbeat information by assuming that the state is not stationary.

Vital detector 218 may perform, for example, the above-described operation on more than one (for example, all) of the detected distance indices f_{b2sel}.

Note that radar apparatus 1 may perform the biological detection by repeating the operation of radar transmitter 100 and the operation of radar receiver 200 a plurality of times, or may perform the same processing as the above-described processing.

As described above, in the present embodiment, radar apparatus 1 controls the transmission of a chirp signal using a first transmission frame and a second transmission frame different from the first transmission frame, and transmits the chirp signal from a transmission antenna. For example, by setting the frame interval (T_{VM}) of the second transmission frame to be shorter than the frame interval (T_{BM}) of the first transmission frame, radar apparatus 1 can detect (track) the walking/body movement in the first transmission frame and can detect vital information of a living body in the second transmission frame. In addition, for example, by setting the transmission period (T_{CB}) of the chirp signal in the first transmission frame to be shorter than the frame interval (T_{VM}) of the second transmission frame and by setting the number of times (N_{CB}) of the transmission of the chirp signal in the first transmission frame to be larger than the number (N_{CV}) of the transmissions of the chirp signal in the second transmission frame, radar apparatus 1 can improve the detection performance (for example, the maximum Doppler frequency or the Doppler resolution) of the walking/body movement in the first transmission frame. In addition, by setting the transmission duration to be shorter than the non-transmission duration in the first transmission frame, the power consumption of radar apparatus 1 can be reduced.

As described above, according to the present embodiment, radar apparatus 1 can achieve both the improvement in the detection accuracy of vital information and the reduction in the heat generation amount and the low power consumption of a radar IC chip.

Note that, in the present embodiment, the case has been described in which the number of transmission antennas used for the transmission in the first transmission frame and the number of transmission antennas used for the transmission in the second transmission frame are the same (for example, see FIG. 5), but the present disclosure is not limited thereto.

For example, the number of transmission antennas used for the transmission in the first transmission frame may be set to be larger than the number of transmission antennas used for the transmission in the second transmission frame. Radar apparatus 1 performs the angle measurement processing in direction estimator 214 using a reception signal in the first transmission frame. By increasing the number of transmission antennas used in the first transmission frame, the number of virtual antennas in the MIMO radar is increased, and the aperture length of the virtual antenna can be enlarged, so that the angle measurement performance in direction estimator 214 can be improved.

Alternatively, for example, the number of transmission antennas used for the transmission in the second transmission frame may be set to be larger than the number of transmission antennas used for the transmission in the first transmission frame. Radar apparatus 1 forms a beam in beam-weight multiplier 217 using a reception signal in the second transmission frame, and then performs the biological detection in vital detector 218. By increasing the number of transmission antennas used in the second transmission frame, the number of virtual antennas in the MIMO radar is increased, the reception SNR of the beam-formed signal in beam-weight multiplier 217 is improved, and the biological detection performance can be improved.

Note that, in the present embodiment, the transmission using a transmission antenna in the first transmission frame or the second transmission frame may be beam transmission in which a plurality of transmission antennas are bundled to form directivity. For example, when the area in which the stationary state is achieved is limited to a specific range in the detection area, the transmission signal transmitted in the second transmission frame may be transmitted by using the beam transmission in which the directivity is directed to the specific range. As a result, the biological detection and the heartbeat detection performance in the stationary state in the specific range can be improved.

### (Variation 1)

In Variation 1, radar apparatus 1 may set the transmission antenna or the number of transmission antennas used for the transmission in the second transmission frame to be variable.

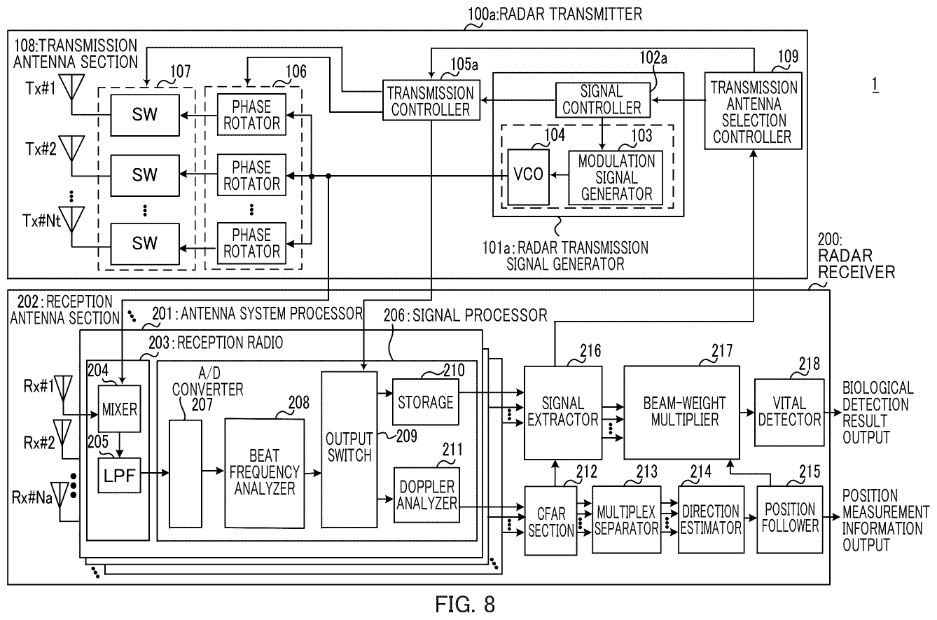

For example, FIG. 8 is a diagram in which transmission antenna selection controller 109 that performs control of adaptively varying the transmission antenna to perform transmission is added to the configuration (for example, FIG. 2) of radar apparatus 1 according to the above-described embodiment. Hereinafter, an operation different from the above-described embodiment will be described with reference to FIG. 8.

The biological detection evaluation index value VDM_{z,n}(f_{b2}) for each transmission antenna calculated in signal extractor 216 is output to transmission antenna selection controller 109.

Transmission antenna selection controller 109 calculates, for example, a transmission antenna biological detection evaluation index value VDₙ(f_{b2}) obtained by summing the plurality of (for example, Na) reception antennas for the n-th transmission antenna as in Expression (33) based on the biological detection evaluation index value VDM_{z,n}(f_{b2}). Then, transmission antenna selection controller 109 may, for example, adaptively select a transmission antenna with an index value exceeding a predetermined value, may select a predetermined number of transmission antennas with index values higher than those of other transmission antennas, or may select a transmission antenna by combining both. Here, z=1 to Na and n=1 to Nt.

$$\begin{matrix}( Expression 33 ) & \\ {VD}_{n} ( {f}_{b 2} ) = {\sum }_{z = 1}^{{N}_{a}} {VDM}_{z , n} ( {f}_{b 2} ) & [ 33 ]\end{matrix}$$

In addition, when a plurality of f_{b2} are included, transmission antenna selection controller 109 may calculate a transmission antenna biological detection evaluation index value VDFₙ for the n-th transmission antenna obtained by summing the plurality of reception antennas including the plurality of f_{b2} as in Expression (34). Then, transmission antenna selection controller 109 may, for example, adaptively select a transmission antenna with an index value exceeding a predetermined value, or may select a predetermined number of transmission antennas with index values higher than those of other transmission antennas. Here, z=1 to Na and n=1 to Nt.

(Expression 34)

VDFₙ=Σ_{f}_{_{b2}}VDₙ(f_{b2}) [34]

Transmission antenna selection controller 109 controls signal controller 102a and transmission controller 105a to set a period (hereinafter, referred to as a “selected transmission antenna transmission duration”) in which the selected transmission antenna is used for transmission in a part of the second transmission frame, for example. For example, transmission antenna selection controller 109 may control signal controller 102a and transmission controller 105a in such a way that, in the second transmission frame of sᵥ=1, the second transmission frame is transmitted using the Nt transmission antennas, and in the second transmission frame of sᵥ=2 to N_{FVAll}, the transmission is performed using the selected transmission antennas whose number is smaller than Nt in the selected transmission antenna transmission duration. Signal controller 102a and transmission controller 105a perform the transmission operation of the second transmission frame using the selected transmission antennas whose number is smaller than Nt, based on the control signal from transmission antenna selection controller 109. The transmission operation is the same as that of the above-described embodiment.

By applying such a selected transmission antenna transmission duration to a part of the transmission periods of the second transmission frame, for example, the transmission ON time can be reduced by reducing the number of transmission antennas in the second transmission frame, and the power consumption of radar apparatus 1 can be further reduced.

### (Variation 2)

In the above-described embodiment, the transmission operation using the first transmission frame and the second transmission frame has been described, but the present disclosure is not limited thereto. For example, control of adaptively switching between a transmission operation that uses the first transmission frame but not the second transmission frame and a transmission operation that uses the second transmission frame but not the first transmission frame may be performed.

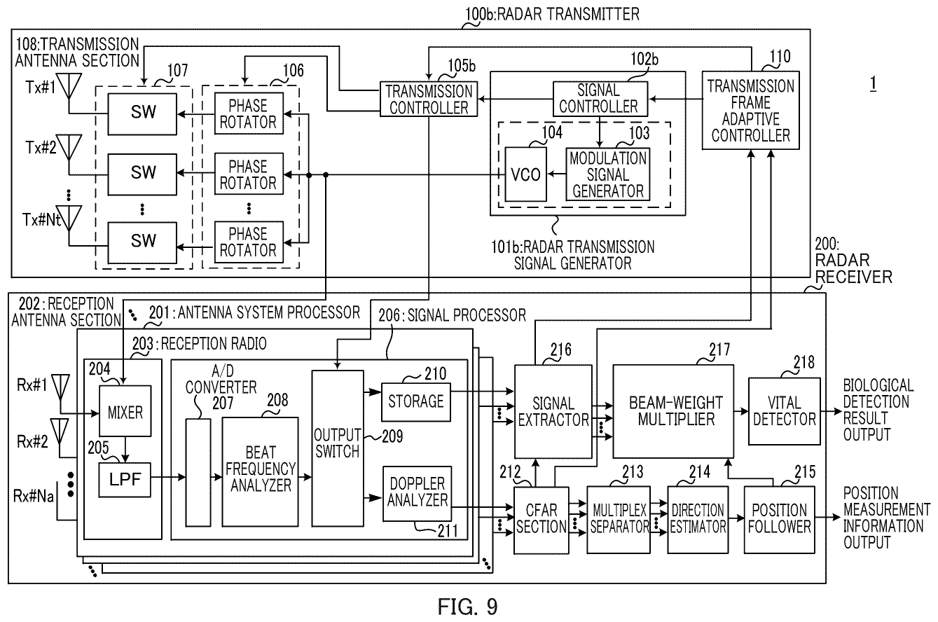

For example, FIG. 9 is a diagram in which transmission frame adaptive controller 110 that performs control of adaptively turning on/off the transmission of the first transmission frame and the second transmission frame is added to the configuration (for example, FIG. 2) of radar apparatus 1 according to the above-described embodiment. Hereinafter, an operation different from the above-described embodiment will be described with reference to FIG. 9.

For example, transmission frame adaptive controller 110 may control signal controller 102b and transmission controller 105b in such a way that, when a stationary object or a moving target object is not detected based on an output of CFAR section 212 that is detected based on transmission signals in the first transmission frame, transmission signals in the second transmission frame is turned off for a predetermined time. As a result, since the transmission signal in the second transmission frame is turned off for a predetermined time, the power consumption of radar apparatus 1 can be further reduced.

In addition, for example, transmission frame adaptive controller 110 may control signal controller 102b and transmission controller 105b in such a way that, when an abrupt change in the body movement is not detected based on the phase difference between plurality of chirp signals based on transmission signals in the second transmission frame in signal extractor 216, transmission signals in the first transmission frame is turned off for a predetermined time.

For example, in the case of transmitting chirp signals with N_{TPA}>1 for each transmission antenna in the second transmission frame, when an abrupt change in the body movement is not detected based on the reception phase difference between the plurality of chirp signals in signal extractor 216, transmission frame adaptive controller 110 sets transmission signals in the first transmission frame to OFF. In addition, at a point in time when an abrupt change in the body movement is detected based on the reception phase difference between the plurality of chirp signals in signal extractor 216, transmission frame adaptive controller 110 may set transmission signals in the first transmission frame to ON.

With such an operation of transmission frame adaptive controller 110, since the transmission signal in the first transmission frame is turned off, the power consumption of radar apparatus 1 can be further reduced.

### (Variation 3)

In the above-described embodiment, the operation example has been described in which vital detector 218 performs the vital detection using a reception signal of the second transmission frame, but the present disclosure is not limited thereto. For example, vital detector 218 may detect the vital information using at least a part of reception signals of the first transmission frame.

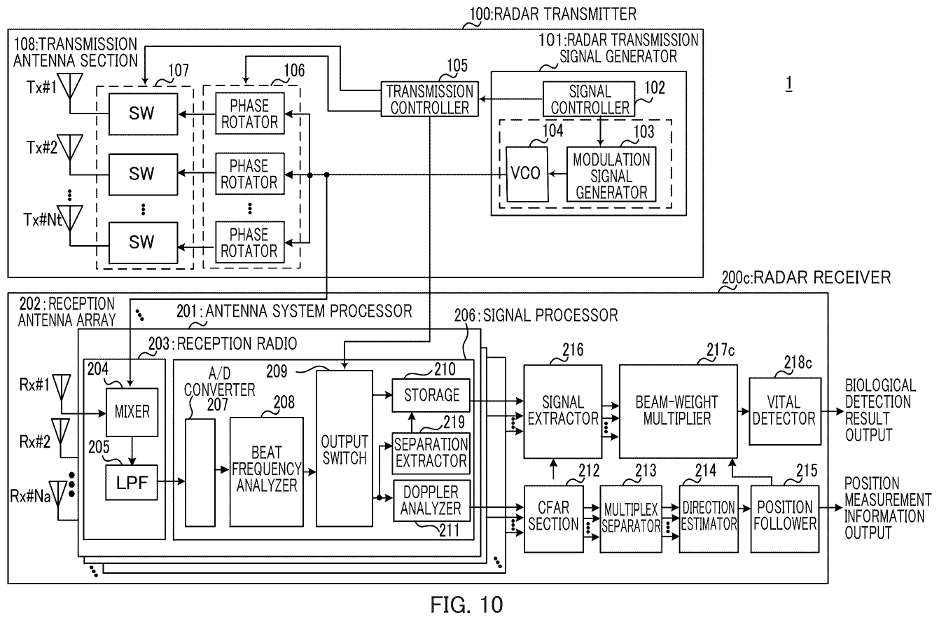

FIG. 10 illustrates a configuration example of radar apparatus 1 according to Variation 3. The configuration in FIG. 10 is different from that of FIG. 2 in that separation extractor 219 is added to the output destination of output switch 209. In addition, in FIG. 10, the output destination of separation extractor 219 is storage 210, and the operations of the components (for example, beam-weight multiplier 217c and vital detector 218c) after storage 210 are partially different from those in the case of FIG. 2.

Output switch 209 switches the output destination of beat frequency analyzer 208 to one of storage 210, Doppler analyzer 211, and separation extractor 219 based on the output control signal from transmission controller 105. For example, the output control signal may be a signal that controls output switch 209 to set the output destination of beat frequency analyzer 208 to Doppler analyzer 211 and separation extractor 219 during the transmission in the first transmission frame and to storage 210 during the transmission in the second transmission frame.

In the case of transmission in the first transmission frame, separation extractor 219 extracts, for example, one or a plurality of beat frequency responses—from beat frequency responses RFT_{z,sb}(f_{b1}, m_{b}) output from beat frequency analyzer 208—near a timing that is an integer multiple of the second transmission frame interval T_{VM} with the start time of the second transmission frame #1 as a reference.

Then, when the simultaneous multiplexing transmission is performed from a plurality of (for example, Nt>1) transmission antennas in the first transmission frame, separation extractor 219 outputs a separated reception signal obtained by separating simultaneous multiplexing transmission signals to storage 210.

Alternatively, when one transmission antenna (Nt=1) is used or time division multiplexing transmission is performed using a plurality of (for example, Nt>1) transmission antennas in the first transmission frame, separation extractor 219 does not need the separation processing, and thus outputs the extracted signal to storage 210.

Here, f_{b1} is a beat frequency index, f_{b1}=0, . . . , N_{data1}/2−1, z=1 to Na, and s_{b}=1 to N_{FB}.

For example, the extraction processing in separation extractor 219 may be performed as follows.

For example, when a is an integer satisfying T_{ofs}−αT_{VM}≥0, and when the number of transmission antennas Nt is 1, separation extractor 219 extracts the beat frequency response RFT_{z,sb}(f_{b1}, m_{bsel0}(α)) with m_{bsel}≤N_{CB} and m_{bsel0}(α)=floor[(T_{ofs}−αT_{VM})/T_{CB}]+1.

In addition, when simultaneous multiplexed signals are transmitted from the plurality of transmission antennas in the first transmission frame, separation extractor 219 extracts beat frequency responses RFT_{z,sb}(f_{b1}, m_{bsel}) for the minimum number of chirp signal transmissions N_{dmul} used for the separation. In this case, m_{bsel}=m_{bsel0}(α), . . . , m_{bsel0}(α)+N_{dmul}−1.

Here, N_{dmul} is, for example, the number of divisions of the phase rotation of 2π in the case of using the Doppler multiplexing transmission, for example, N_{DM} in the case of using Expression (6), and N_{DM}+Nᵢₙₜ in the case of using Expression (7). In addition, N_{dmul} is, for example, the code length Loc used for the code multiplexing in the case of using the code multiplexing transmission.

In addition, when time-division multiplexed signals are transmitted from the plurality of (Nt) transmission antennas in the first transmission frame, separation extractor 219 extracts, for example, the beat frequency responses RFT_{z,sb}(f_{b1}, m_{bsel}) with the transmission antennas N_{dmul}=Nt. For example, m_{bsel}=m_{bsel0}(α), . . . , m_{bsel0}(α)+Nₜ−1.

Note that N_{dmul}<Nt may be established.

The separation processing in separation extractor 219 may be performed as follows.

Separation extractor 219 separates the multiplexed signals by imparting a conjugate phase rotation to the phase rotation (in the case of the code multiplexing, the code) imparted for each transmission antenna.

For example, when the Doppler multiplexing transmission is performed in which the phase shift amount ψ_{ndm}(m_{b}) as in Expression (8) is imparted to a chirp signal for the n_{dm}-th transmission antenna, separation extractor 219 performs the separation processing as in Expression (35) (here, the multiplexed signal separation processing assuming a stationary object is performed).

$$\begin{matrix}( Expression 35 ) & \\ {DmulRFT}_{z , {s}_{b}} ( {f}_{b 1} , ndm ) = {\sum }_{ndmul = 0}^{{N}_{dmul} - 1} {RFT}_{z , {s}_{b}} ( {f}_{b 1} , {m}_{bsel 0} + ndmul ) exp [ - j {ψ}_{ndm} ( {m}_{bsel 0} + ndmul ) ] & [ 35 ]\end{matrix}$$

Similarly, in the case of the code multiplexing, for example, when the n_{code}-th code is imparted for a transmission antenna (in the case of performing the code multiplexing transmission in which the phase shift amount (for example, ψ_{1,ncode}, ψ_{2,ncode}, . . . , and ψ_{Ndmul,ncode}) corresponding to the n_{code}-th code element is imparted to a chirp signal for each transmission period), separation extractor 219 performs the separation processing as in Expression (36) (here, the multiplexed signal separation processing assuming a stationary object is performed). Here, ncode=1 to Nt.

$$\begin{matrix}( Expression 36 ) & \\ {DmulRFT}_{z , {s}_{b}} ( {f}_{b 1} , ncode ) = {\sum }_{ndmul = 0}^{{N}_{dmul} - 1} {RFT}_{z , {s}_{b}} ( {f}_{b 1} , {m}_{bsel 0} + ndmul ) exp [ - j {ψ}_{ndmul + 1 , ncode} ] & [ 36 ]\end{matrix}$$

Hereinafter, a signal extracted in separation extractor 219 will be represented by “DMO_{z,n}(f_{b1}, s_{b}, α)”. Here, n=1 to Nt, and α is an integer α satisfying T_{ofs}−αT_{VM}≥0. In addition, f_{b1} is a beat frequency index, f_{b1}=0, . . . , N_{data1}/2−1, z=1 to Na, and s_{b}=1 to N_{FB}.

Storage 210 temporarily stores the output DMO_{z,n}(f_{b1}, s_{b}, α) from separation extractor 219, in addition to the operation of the above-described embodiment.

Beam-weight multiplier 217c performs the following operation, in addition to the operation of the above-described embodiment.

When the output DMO_{z,n}(f_{b1sel}, s_{b}, α) of separation extractor 219 includes reception signals by the plurality of transmission antennas or the plurality of reception antennas, beam-weight multiplier 217c multiplies the output by a beam weight that improves the reception SNR and outputs the result to vital detector 218c.

Here, f_{b1sel} represents a distance index f_{b1} that is closest to the distance indicated by the extracted distance index f_{b2sel} in the output Y_{z,n}(f_{b2sel}, mₜₛₑₗ) from signal extractor 216. For example, f_{b1sel} may represent a distance index that matches or substantially matches the distance information R(f_{b2}) indicated by the distance index f_{b2sel} (for example, R(f_{b1sel})≈R(f_{b2sel})) or a plurality of distance indices that are within a predetermined distance range including the distance information R(f_{b2sel}). For example, R(f_{b2sel})−Rb≤R(f_{b1sel})≤R(f_{b2sel})+Rb, and Rb represents a predetermined distance.

Beam-weight multiplier 217c multiplies the output DMO_{z,n}(f_{b1}, s_{b}, α) of separation extractor 219 of the first transmission frame by the beam weight W(f_{b2sel}) in the same manner. The output is close to mₜₛₑₗ, namely an index number (for example, the serial number at the unit of biological processing) of the second transmission frame, in the output Y_{z,n}(f_{b2sel}, mₜₛₑₗ) from signal extractor 216.

Note that, when transmission antennas are different between the first transmission frame and the second transmission frame, beam-weight multiplier 217c may extract a value corresponding to the same transmission antenna as the transmission antenna used in the second transmission frame from the beam weight W(f_{b2sel}) and perform the multiplication processing on the extracted value. For example, in the case of FIG. 10, mₜₛₑₗ may be associated as s_{b}=floor[(mₜₛₑₗ−1)/Nfv]+1. Therefore, the reception signal DMO_{z,n}(f_{b1sel}, floor[(mₜₛₑₗ−1)/Nfv]+1, α) between the first transmission frames includes the phase information reflecting the displacement status of a body surface when body surface is observed at the T_{VM} cycle in a period in which the second transmission frame is not transmitted (a period between mod(mₜₛₑₗ, Nfv)=0 and mod(mₜₛₑₗ, Nfv)=1). For example, beam-weight multiplier 217c outputs, as an interpolation beam weight multiplication signal in a period between mod(mₜₛₑₗ, Nfv)=0 and mod(mₜₛₑₗ, Nfv)=1, a signal obtained by multiplying the output DMO_{z,n}(f_{b1sel}, s_{b}, α) of separation extractor 219 of the first transmission frame by the beam weight W(f_{b2sel}) in the same manner as in Expression (37) and Expression (38) to vital detector 218c.

$$\begin{matrix}( Expression 37 ) & \\ BO ( {f}_{b 2 sel} , {m}_{tsel} , α ) = {W ( {f}_{b 2 sel} )}^{H} z ( {f}_{b 1 sel} , floor [ \frac{{m}_{tsel}}{{N}_{fv}} ] , α ) & [ 37 ]\end{matrix}$$

$$\begin{matrix}( Expression 38 ) & \\ z ( {f}_{b 1 sel} , {s}_{b} , α ) = [ \begin{matrix}{DMO}_{1 , 1} ( {f}_{b 1 sel} , {s}_{b} , α ) \\ {DMO}_{2 , 1} ( {f}_{b 1 sel} , {s}_{b} , α ) \\ ⋮ \\ {DMO}_{Na , 1} ( {f}_{b 1 sel} , {s}_{b} , α ) \\ {DMO}_{1 , 2} ( {f}_{b 1 sel} , {s}_{b} , α ) \\ {DMO}_{2 , 2} ( {f}_{b 1 sel} , {s}_{b} , α ) \\ ⋮ \\ {DMO}_{Na , 2} ( {f}_{b 1 sel} , {s}_{b} , α ) \\ ⋮ \\ {DMO}_{1 , Nt} ( {f}_{b 1 sel} , {s}_{b} , α ) \\ {DMO}_{2 , Nt} ( {f}_{b 1 sel} , {s}_{b} , α ) \\ ⋮ \\ {DMO}_{Na , Nt} ( {f}_{b 1 sel} , {s}_{b} , α )\end{matrix} ] & [ 38 ]\end{matrix}$$

Vital detector 218c performs heartbeat detection in a similar manner to the operation example of the above-described embodiment in addition to the operation of the above-described embodiment as follows: using, as an interpolation value, an interpolation beam weight multiplication signal calculated by beam-weight multiplier 217c as the phase information reflecting the displacement status of a body surface when body surface is observed at the T_{VM} cycle in a period in which the second transmission frame is not transmitted during the transmission duration of the first transmission frame (a period between mod(mₜₛₑₗ, N_{fv})=0 and mod(mₜₛₑₗ, N_{fv})=1).

### (Version 4)

Radar apparatus 1 according to Variation 4 may have the same configuration as radar apparatus 1 according to Variation 3 (FIG. 10), for example.

In Variation 3, the operation example has been described in which vital detector 218c in radar apparatus 1 detects vital information including a reception signal of the second transmission frame and a reception signal of the first transmission frame, but the present disclosure is not limited thereto. For example, the same vital information can be detected using a transmission signal different from the transmission signal for the walking/body movement detection included in the transmission signals of the first transmission frame.

Hereinafter, an example will be described in which, in the first transmission frame, a transmission signal that is the same as (identical or common with) a transmission signal in the second transmission frame is transmitted in addition to a transmission signal for the walking/body movement detection.

By transmitting a transmission signal the same as that in the second transmission frame used for the vital detection in the first transmission frame, the influence of the reception phase variation due to the difference in transmission signals can be reduced, and the accuracy of the heartbeat detection can be improved.

### Transmission Example 1

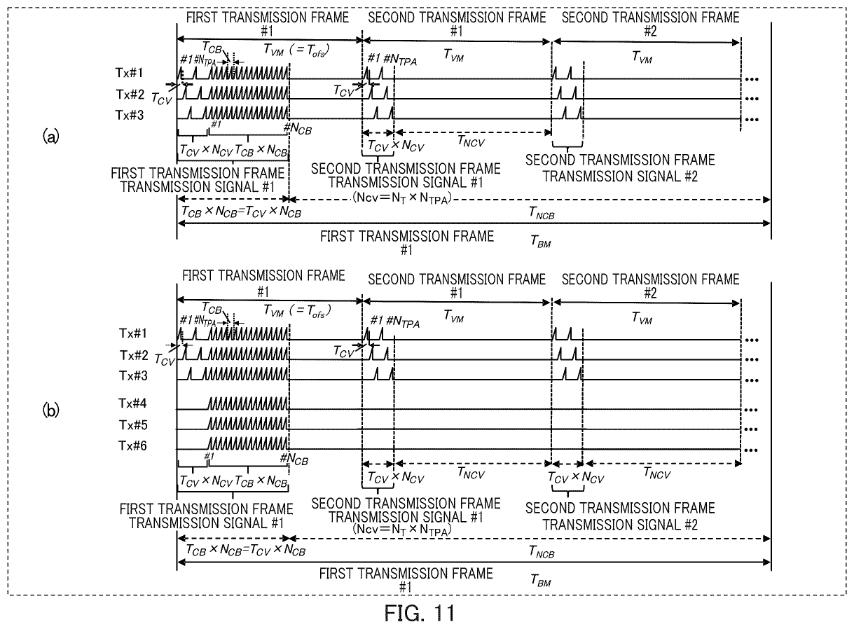

An operation example of each component of radar transmitter 100 according to the transmission example 1 will be described with reference to FIG. 11. FIG. 11 illustrates radar transmission signals when the number of transmission antennas Nt is 3 as an example. In FIG. 11, the vertical axis for each transmission antenna is a transmission frequency, and a sawtooth-shaped signal portion represents chirp signals. In addition, chirp signals transmitted simultaneously or time-divisionally from the transmission antennas indicate the case where the chirp signals having the same center frequency f_{c} and the same transmission bandwidth B_{w} are transmitted, but the transmitted chirp signals do not need to have the same center frequency f_{c} or the same transmission bandwidth B_{w}.

FIG. 11 illustrates an example in which a transmission signal the same as that in the second transmission frame is transmitted at a timing different from a transmission signal for the walking/body movement detection in the first transmission frame.

In addition, part (a) in FIG. 11 illustrates an example in which the number of transmission antennas for transmission signals for the walking/body movement detection in the first transmission frame is 3 (Nₜ₁=3) and is the same as the number of transmission antennas in the second transmission frame (Nₜ₂=3).

In addition, part (b) in FIG. 11 illustrates an example in which the number of transmission antennas for transmission signals for the walking/body movement detection in the first transmission frame is 6 (Nₜ₁=6) and is different from the number of transmission antennas in the second transmission frame (Nₜ₂=3).

Note that the transmission of a chirp signal of the second transmission frame is started after T_{ofs} has elapsed from the start timing of the first chirp signal of the first transmission frame, and in the example of FIG. 11, T_{ofs}=T_{VM} is set, but the present disclosure is not limited thereto. T_{ofs} does not have to be equal to T_{VM}.

Signal controller 102 controls modulation signal generator 103 and VCO 104 to generate radar transmission signals (chirp signals) including a first transmission frame and a second transmission frame as follows. Here, the second transmission frame is the same transmission frame as that of the above-described embodiment, and the description thereof will be omitted.

The first transmission frame includes a chirp transmission duration (=T_{CB}×N_{CB}) in which the chirp signal is transmitted N_{CB} times at a transmission period (T_{CB}) and a chirp transmission duration (=T_{CV}×N_{CV}) in which a chirp signal the same as in the second transmission frame is transmitted.

The transmission signals and the transmission operation in the transmission duration of T_{CB}×N_{CB} are the same as those of the above-described embodiment, and the description thereof will be omitted. In addition, the transmission operation of the transmission signals (chirp signals the same as in the second transmission frame) in the transmission duration of T_{CV}×N_{CV} is the same as that of the above-described embodiment, and the description thereof will be omitted.

As illustrated in parts (a) and (b) in FIG. 11, in the first transmission frame, a transmission signal (for example, a signal for estimating the walking/body movement) in the transmission duration of T_{CB}×N_{CB} and a transmission signal (for example, a transmission signal the same as in the second transmission frame) in the transmission duration of T_{CV}×N_{CV} are transmitted at different timings. In addition, as illustrated in parts (a) and (b) in FIG. 11, in the first transmission frame, transmission signals in the transmission duration of T_{CB}×N_{CB} are subjected to simultaneous multiplexing transmission using a plurality of transmission antennas, and transmission signals in the transmission duration of T_{CV}×N_{CV} are subjected to time division multiplexing using a plurality of transmission antennas.

Hereinafter, an example of transmission signals in the transmission duration of T_{CV}×N_{CV} will be described.

The example of FIG. 11 illustrates the case where the chirp transmission duration (=T_{CV}×N_{CV}) in which a chirp signal the same as in the second transmission frame is transmitted is set to a timing before the chirp transmission duration (=T_{CB}×N_{CB}) in which a chirp signal is transmitted N_{CB} times at the transmission period (T_{CB}), but the present disclosure is not limited thereto. For example, the same effect can be obtained when the transmission duration of T_{CV}×N_{CV} is set to a timing after a chirp transmission duration (=T_{CB}×N_{CB}) in which a chirp signal is transmitted N_{CB} times at the transmission period (T_{CB}). For example, T_{ofs}=T_{CB}×N_{CB}+T_{VM} may be set.

Signal controller 102 controls modulation signal generator 103 and VCO 104 in such a way that, for the chirp transmission duration (=T_{CV}×N_{CV}) in which a chirp signal the same as in the second transmission frame is transmitted, a timing near an integer multiple of the second transmission frame interval T_{VM} is set as the start timing, with the start time of the second transmission frame as a reference.

Note that, for example, when the first transmission frame T_{ofs}≥2×T_{VM} is set, the first transmission frame may include a plurality (=floor(T_{ofs}/T_{VM}) times) of chirp transmission durations (=T_{CV}×N_{CV}) in which a chirp signal the same as in the second transmission frame is transmitted (not illustrated). As a result, for example, by continuously transmitting a chirp signal the same as in the second transmission frame in the first transmission frame, the influence of the reception phase variation due to the difference in transmission signals can be reduced for the reception phase variation at the plurality of second transmission frame intervals T_{VM} in the first transmission frame, and the detection accuracy of the vital information can be improved.

Next, an operation example of radar receiver 200 will be described.

In Variation 4, radar receiver 200 detects vital information in vital detector 218c using a reception signal of the first transmission frame in addition to a reception signal of the second transmission frame, as in the operation example of radar receiver 200 in Variation 3. Hereinafter, an operation different from Variation 3 will be described.

From beat frequency responses output from beat frequency analyzer 208 during the transmission in the first transmission frame, separation extractor 219 extracts one or a plurality of beat frequency responses near a timing that is an integer multiple of the second transmission frame interval T_{VM} with the start time of the second transmission frame #1 as a reference. Then, when the time division multiplexing transmission is performed using a plurality of (for example, Nt>1) transmission antennas in the first transmission frame as in the second transmission frame, separation extractor 219 does not need the separation processing, and thus outputs the extracted signal to storage 210.

Since a chirp signal the same as in the second transmission frame is transmitted in the first transmission frame, the beat frequency response RFT_{z,sb}(f_{b1}, m_{b}) can also be represented by “beat frequency response RFT_{z,sb}(f_{b2}, m_{b})”. For example, the distance information indicated by the beat frequency index is the same as the distance information in the second transmission frame. Here, f_{b2} is a beat frequency index, f_{b2}=0, . . . , N_{data2}/2−1, z=1 to Na, and s_{b}=1 to N_{FB}.

Here, when a is an integer satisfying T_{ofs}−αT_{VM}≥0, and when the time division multiplexing transmission is performed using a plurality of (Nt>1) transmission antennas in the first transmission frame as in the second transmission frame, separation extractor 219 extracts the beat frequency response RFT_{z,sb}(f_{b2}, m_{bsel0}(α)) in which m_{bsel}≤N_{CB} and m_{bsel0}(α)=floor[(T_{ofs}−αT_{VM})/T_{CB}]+1.

Hereinafter, a signal extracted in separation extractor 219 will be represented by “DMO_{z,n}(f_{b2}, s_{b}, α)”. Here, n=1 to Nₜ, and α is an integer α satisfying T_{ofs}−αT_{VM}≥0. In addition, f_{b2} is a beat frequency index, f_{b2}=0, . . . , N_{data2}/2−1, z=1 to Na, and s_{b}=1 to N_{FB}.

The operations of beam-weight multiplier 217c and vital detector 218c hereinafter may be the same as in the operation example of Variation 3.

With the above-described operation, as in Variation 3, vital detector 218c can detect, by using a signal transmitted in the same manner as the second transmission frame, an interpolation beam weight multiplication signal calculated by beam-weight multiplier 217c as the phase information reflecting the displacement status of a body surface when the body surface is observed at the T_{VM} cycle in a period in which the second transmission frame is not transmitted during the transmission duration of the first transmission frame (for example, a period between mod(mₜₛₑₗ, N_{fv})=0 and mod(mₜₛₑₗ, N_{fv})=1). Therefore, radar apparatus 1 can reduce the influence of the reception phase variation due to the difference in transmission signals, and can improve the accuracy of the vital detection.

### Transmission Example 2

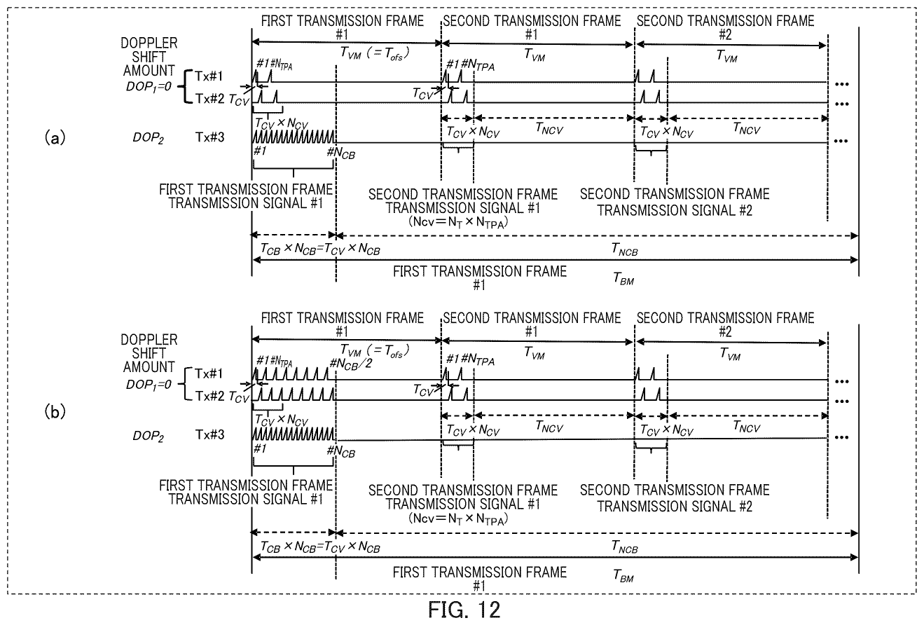

An operation example of each component of radar transmitter 100 according to the transmission example 2 will be described with reference to FIG. 12. In FIG. 12, the vertical axis for each transmission antenna is a transmission frequency, and a sawtooth-shaped signal portion represents chirp signals. In addition, the chirp signals transmitted simultaneously or time-divisionally from the transmission antennas indicate the case where the chirp signals having the same center frequency f_{c} and the same transmission bandwidth B_{w} are transmitted, but the transmitted chirp signals do not need to have the same center frequency f_{c} or the same transmission bandwidth B_{w}. Note that, hereinafter, the number of transmission antennas in the first transmission frame and the second transmission frame will be represented by Nₜ₁ and Nₜ₂, respectively. FIG. 12 illustrates radar transmission signals when the number of transmission antennas Nₜ₁ in the first transmission frame is 3 and the number of transmission antennas Nₜ₂ in the second transmission frame is 2 as an example.

In FIG. 12, in the first transmission frame, a transmission signal for the walking/body movement detection mainly used for the walking/body movement detection is transmitted using the transmission antenna Tx #3 among the Nₜ₁ transmission antennas (Nₜ₁=3). In addition, in the first transmission frame in FIG. 12, a transmission signal the same as the transmission signal in the second transmission frame is transmitted by a transmission antenna (Tx #1, 2 in FIG. 12) the same as in the second transmission frame by using a Doppler multiplexed signal different from the transmission signal for the walking/body movement detection. As described above, in the first transmission frame, radar apparatus 1 performs, using different Doppler shift amounts, Doppler multiplexing transmission on a transmission signal for the walking/body movement detection and on a transmission signal (for example, a transmission signal the same as in the second transmission frame) different from the transmission signal for the walking/body movement detection.

Part (a) in FIG. 12 illustrates an example in which a transmission signal the same as the transmission signal in the second transmission frame is transmitted by a transmission antenna (Tx #1, 2 in FIG. 12) the same as in the second transmission frame for a period of (T_{CV}×Ncv) in the first transmission frame and then the transmission is stopped.

In addition, part (b) in FIG. 12 illustrates an example in which a transmission signal the same as the transmission signal in the second transmission frame is transmitted by a transmission antenna (Tx #1, 2 in FIG. 12) the same as in the second transmission frame for a period of (Tcv×Ncv) in the first transmission frame, and the same signal is continuously transmitted for a period of (T_{CV}×N_{CB}) without stopping the transmission. In the case of part (b) in FIG. 12, radar apparatus 1 can use the signal continuously transmitted for a period of (T_{CV}×N_{CB}) also for the transmission signal for the walking/body movement detection, so that the number of transmission antennas for the walking/body movement detection can be increased. In part (b) in FIG. 12, radar apparatus 1 can perform the walking/body movement detection including Tx #1 and 2 in addition to Tx #3, and can improve the detection performance of the walking/body movement detection.

For example, the transmission of a chirp signal of the second transmission frame is started after T_{ofs} has elapsed from the start timing of the first chirp signal of the first transmission frame. In the example of FIG. 12, T_{ofs}=T_{VM} is set, but the present disclosure is not limited thereto. T_{ofs} does not have to be equal to T_{VM}.

Signal controller 102 controls modulation signal generator 103 and VCO 104 to generate radar transmission signals (chirp signals) composed of a first transmission frame and a second transmission frame as follows. Here, the second transmission frame is the same transmission frame as that of the above-described embodiment, and the description thereof will be omitted.

In addition, in the first transmission frame, a transmission signal for the walking/body movement detection and a transmission signal the same as the transmission signal in the second transmission frame are subjected to simultaneous multiplexing transmission by the Doppler multiplexing. Therefore, for example, a chirp signal in the first transmission frame may be generated by the modulation parameters that are all the same as for a chirp signal in the second transmission frame. For example, hereinafter, T_{CB}=T_{CV} is established.

Signal controller 102 controls modulation signal generator 103 and VCO 104 in such a way that, for the chirp transmission duration (=T_{CV}×N_{CV}) in which a chirp signal the same as in the second transmission frame is transmitted, a timing near an integer multiple of the second transmission frame interval T_{VM} with the start time of the second transmission frame as a reference is set as the start timing.

Note that, for example, when the first transmission frame T_{ofs}≥2×T_{VM} is set, the first transmission frame may include a plurality (=floor(T_{ofs}/T_{VM}) times) of the chirp transmission durations (=T_{CV}×N_{CV}) in which a chirp signal the same as in the second transmission frame is transmitted (not illustrated). As a result, for example, by continuously transmitting a chirp signal the same as in the second transmission frame in the first transmission frame, the influence of the reception phase variation due to the difference in transmission signals can be reduced for the reception phase variation at the plurality of second transmission frame intervals T_{VM} in the first transmission frame, and the detection accuracy of the vital information can be improved.

In the first transmission frame, the chirp signal is transmitted at a transmission period (T_{CB}=T_{CV}). In the first transmission frame, during a chirp transmission duration of N_{CB} times (=T_{CV}×N_{CB}) transmission, chirp signals the same as in the second transmission frame are transmitted for a period equal to or longer than the chirp transmission duration (=T_{CV}×N_{CV}) and including a period in which the chirp signals are subjected to simultaneous multiplexing transmission within the T_{CV}×N_{CB} period. As described above, in the first transmission frame, the transmission ON time of a chirp signal can be reduced by performing simultaneous multiplexing transmission on a transmission signal mainly for detecting the walking/body movement and on a transmission signal the same as in the second frame.

In part (a) in FIG. 12, in the first transmission frame, the chirp signal is transmitted at a transmission period (T_{CB}=T_{CV}). In addition, in part (a) in FIG. 12, the chirp transmission duration (=T_{CV}×N_{CB}) in which a chirp signal is transmitted N_{CB} times includes a period in which a chirp signal the same as in the second transmission frame is Doppler multiplexed and simultaneously transmitted in a chirp transmission duration (T_{CV}×N_{CV}).

In addition, in part (b) in FIG. 12, in the first transmission frame, a chirp signal is transmitted at a transmission period (T_{CB}=T_{CV}). In addition, in part (b) in FIG. 12, a period in which a chirp signal the same as in the second transmission frame is transmitted is configured to include a period in which the chirp signal is Doppler multiplexed and simultaneously transmitted to match the chirp transmission duration (=T_{CV}×N_{CB}) in which the chirp signal is transmitted N_{CB} times. In this case, radar apparatus 1 can detect the walking/body movement using reception signals corresponding to Tx #1 and Tx #2 in addition to Tx #3 illustrated in part (b) in FIG. 12.

Note that, for example, in the case of T_{ofs}≥2T_{VM} in the first transmission frame and 2T_{VM}≥T_{CV}×N_{CB}, a transmission duration in which a chirp signal the same as in the second transmission frame is transmitted without performing Doppler multiplexing transmission may be included.

Here, the Doppler multiplexed signal of a transmission signal the same as the transmission signal of the second transmission frame in the first transmission frame may be transmitted using a Doppler multiplexed signal (Doppler shift amount DOP₁) the same as the transmission signal of the second transmission frame. On the other hand, a transmission signal in the chirp transmission duration (=T_{CB}×N_{CB}) in which a chirp signal is transmitted at the transmission period (T_{CB}) and transmitted N_{CB} times may be Doppler-multiplexed using a Doppler shift amount different from DOP₁.

In the first transmission frame, transmission controller 105 imparts the following phase rotation to phase rotator 106 using a phase control signal in order to perform the Doppler multiplexing as described above.

A transmission signal the same as the transmission signal of the second transmission frame may be assigned one Doppler multiplexed signal DOP₁ since the transmission signal is time-divisionally transmitted from a part of the Nt transmission antennas (Tx #1, 2 in the example of FIG. 12). Here, since the Doppler multiplexed phase rotation is not imparted to the transmission signal of the second transmission frame, the Doppler shift amount can be regarded as zero. Therefore, for example, a Doppler shift amount DOP₁=0 the same as the second transmission frame may be assigned to a transmission signal in the first transmission frame the same as the transmission signal of the second transmission frame.

On the other hand, a transmission signal in the chirp transmission period (=T_{CB}×N_{CB}) in which a chirp signal is transmitted at the transmission duration (T_{CB}=T_{CV}) and transmitted N_{CB} times may be Doppler-multiplexed using a Doppler multiplexed signal (for example, a Doppler shift amount) different from the signal in the second transmission frame. In the example of FIG. 12, since the Doppler multiplexing is performed using the transmission antenna Tx #3, a Doppler shift amount DOP₂ different from that of the second transmission frame (or Tx #1, Tx #2) is imparted.

Transmission controller 105 may apply even interval Doppler shift amount setting (for example, Expression (6)) or uneven interval Doppler shift amount setting (for example, Expression (7)) with N_{DM}=2 (=Nt₁−Nt₂+1), and impart a predetermined phase rotation to phase rotator 106 using a phase control signal.

Note that, since the Doppler multiplexed signals include time-division multiplexed signals of the transmission antennas of Nₜ₂=2, the phase rotation using Expression (39) instead of Expression (8) may be imparted to be the same phase rotation in Nₜ₂ chirp transmission durations.

$$\begin{matrix}( Expression 39 ) & \\ {ψ}_{ndm} ( {m}_{b} ) = floor [ \frac{{m}_{b} + 1}{{N}_{t 2}} ] \times {ϕ}_{ndm} & [ 39 ]\end{matrix}$$

The outputs (for example, referred to as Doppler multiplexed signals (DDM signals)) from the Nt phase rotators 106 are amplified to predetermined transmission power through SW 107 and are radiated into space from the Nt transmission antennas of transmission antenna section 108, respectively.

Here, in the example of FIG. 12, transmission controller 105 outputs antenna switching control signals to SW 107, which include setting corresponding to the time-division switching, so as to sequentially achieve the output ON state in order to transmit a time-division manner from the transmission antennas Tx #1, 2 among the Nt transmission antennas. Note that transmission controller 105 sets and outputs the antenna switching control signals so as to sequentially achieve the output ON state in response to the time-division switching in the chirp transmission duration (in the case of FIG. 12, T_{CV}×N_{CB}) in which a chirp signal the same as in the second transmission frame is Doppler-multiplexed and simultaneously transmitted, and the output OFF state is achieved in other periods.

In addition, in the example of FIG. 12, since a chirp signal is Doppler-multiplexed and transmitted from the transmission antenna Tx #3, transmission controller 105 sets and outputs the antenna switching control signal in such a way that SW 107 connected to the transmission antenna Tx #3 is turned on in the first transmission frame transmission duration (=T_{CV}×N_{CB}).

Next, an operation example of radar receiver 200 will be described.

In Variation 4, radar receiver 200 detects vital information in vital detector 218c using a reception signal of the first transmission frame in addition to the second transmission frame, as in the operation example of radar receiver 200 in Variation 3. Hereinafter, an operation different from Variation 3 will be described.

Separation extractor 219 extracts, from the beat frequency response outputs from beat frequency analyzer 208 during the transmission in the first transmission frame, one or a plurality of beat frequency responses near a timing that is an integer multiple of the second transmission frame interval T_{VM} with the start time of the second transmission frame #1 as a reference. Then, separation extractor 219 outputs to storage 210 the separated reception signals that are obtained by separating the signals having been subjected to time division multiplexing transmission using the Nt₂ transmission antennas and Doppler-multiplexed and simultaneously transmitted using N_{DM} (=Nt₁−Nt₂+1) in the first transmission frame.

Since a chirp signal the same as in the second transmission frame is transmitted in the first transmission frame, the beat frequency response RFT_{z,sb}(f_{b1}, m_{b}) can also be represented by “beat frequency response RFT_{z,sb}(f_{b2}, m_{b})”. For example, the distance information indicated by the beat frequency index is the same as the distance information in the second transmission frame. Here, f_{b2} is a beat frequency index, f_{b2}=0, . . . , N_{data2}/2−1, z=1 to Na, and s_{b}=1 to N_{FB}.

Here, separation extractor 219 extracts the beat frequency response RFT_{z,sb}(f_{b2}, m_{bsel0}(α)) in which α is an integer satisfying T_{ofs}−αT_{VM}≥0, m_{bsel}≤N_{CB}, and m_{bsel0}(α)=floor[(T_{ofs}−αT_{VM})/T_{CB}]+1. In addition, when the time division multiplexing transmission is performed using Nₜ₂ (e.g., two or more) transmission antennas and Doppler multiplexed signals are transmitted using Nₜ₁−Nₜ₂ transmission antennas in the first transmission frame as in the second transmission frame, separation extractor 219 extracts the beat frequency response of Nₜ₂×N_{dmul} obtained by multiplying the minimum number of chirp signal transmissions N_{dmul} used for the separation by the number of time divisions. In this case, m_{bsel}=m_{bsel0}(α), . . . , m_{bsel0}(α)+Nₜ₂×N_{dmul}−1.

Here, N_{dmul} is, for example, the number of divisions of the phase rotation of 2π in the case of using the Doppler multiplexing transmission, for example, N_{DM}=2 in the case of using Expression (6), and N_{DM}+Nᵢₙₜ in the case of using Expression (7).

The separation processing in separation extractor 219 may be performed as follows.

Separation extractor 219 separates multiplexed signals by imparting a conjugate phase rotation to the phase rotation (in the case of the code multiplexing, the code) imparted for each transmission antenna.

For example, when time-division multiplexed signals of the Nₜ₂ transmission antennas are included in Doppler multiplexed signals for the n_{dm}-th transmission antenna and the same phase rotation is imparted in Nₜ₂ chirp transmission durations (for example, in the case of performing Doppler multiplexing transmission in which the phase shift amount ψ_{ndm}(m_{b}) as in Expression (39) is imparted to a chirp signal and output), separation extractor 219 performs the separation processing in which the time-division multiplexing and the Doppler multiplexing are considered as in Expression (40) (here, the multiplexed signal separation processing assuming a stationary object is performed). Here, ndm is a Doppler multiplexing index, and ndm=1 to N_{DM}. In addition, ntdm is a time division multiplexing index, and ntdm=1 to Nₜ₂.

$$\begin{matrix}( Expression 40 ) & \\ {DmulRFT}_{z , {s}_{b}} ( {f}_{b 1} , ndm , ntdm ) = {\sum }_{ndmul = 0}^{{N}_{dmul} - 1} {RFT}_{z , {s}_{b}} ( {f}_{b 1} , {m}_{bsel 0} + {N}_{t 2} \times ndmul + ntdm - 1 ) exp [ - j {ψ}_{ndm} ( {m}_{bsel 0} + {N}_{t 2} \times ndmul + ntdm - 1 ) ] & [ 40 ]\end{matrix}$$

For example, in the example of FIG. 12, the separation signal for each transmission antenna is as follows. Tx #1 is DmulRFT_{z,sb}(f_{b1}, 1, 1) with ndm=1 and ntdm=1, Tx #2 is DmulRFT_{z,sb}(f_{b1}, 1, 2) with ndm=1 and ntdm=2, and Tx #3 is DmulRFT_{z,sb}(f_{b1}, 2, 1) and DmulRFT_{z,sb}(f_{b1}, 2, 2) with ndm=2 and ntdm=1, 2.

Hereinafter, a signal extracted in separation extractor 219 will be represented by “DMO_{z,n}(f_{b2}, s_{b}, α)”. Here, n=1 to Nₜ, and α is an integer α satisfying T_{ofs}−αT_{VM}≥0. In addition, f_{b2} is a beat frequency index, f_{b2}=0, . . . , N_{data2}/2−1, z=1 to Na, and s_{b}=1 to N_{FB}.

The subsequent operations of beam-weight multiplier 217c and vital detector 218c may be the same as the operations in Variation 3.

With the above-described operation, vital detector 218c can detect, by using a signal transmitted in the same manner as the second transmission frame as in Variation 3, an interpolation beam weight multiplication signal calculated by beam-weight multiplier 217c as the phase information reflecting the displacement status of a body surface when the body surface is observed at the T_{VM} cycle in a period in which the second transmission frame is not transmitted during the transmission duration of the first transmission frame (for example, a period between mod(mₜₛₑₗ, Nfv)=0 and mod(mₜₛₑₗ, Nfv)=1). Therefore, radar apparatus 1 can reduce the influence of the reception phase variation due to the difference in transmission signals, and can improve the accuracy of the vital detection.

Note that, since the Doppler multiplexed signals include the time-division multiplexed signals of the Nₜ₂ transmission antennas (Nₜ₂=2), the phase rotation using Expression (39) is imparted to be the same phase rotation in Nₜ₂ chirp transmission durations. Therefore, Doppler analyzer 211 of radar receiver 200 performs the Doppler analysis for each time-division multiplexed reception signal, and thus performs the Doppler analysis using the Nₜ₂ Doppler analyzers 211.

For example, the output “VFT_{z,sb,ntdm}(f_{b1}, fₛ)” of the ntdm-th Doppler analyzer 211 in z-th signal processor 206 in the s_{b}-th first transmission frame is shown in Expression (41). Note that j is an imaginary unit, and z=1 to Na and s_{b}=1 to N_{FB}. In addition, ntdm=1 to Nₜ₂.

$$\begin{matrix}( Expression 41 ) & \\ {VFT}_{z , {s}_{b} , ntdm} ( {f}_{b 1} , {f}_{s} ) = {\sum }_{u = 0}^{\frac{{N}_{CB}}{{N}_{t 2}} - 1} {RFT}_{z , {s}_{b}} ( {f}_{b 1} , {N}_{t 2} \times u + ntdm ) exp [ - j \frac{2 π {uf}_{s}}{( \frac{{N}_{CB}}{{N}_{t 2}} )} ] {VFT}_{z , {s}_{b} , ntdm} ( {f}_{b 1} , {f}_{s} ) = {\sum }_{u = 0}^{\frac{{N}_{CB}}{{N}_{t 2}} - 1} {RFT}_{z , {s}_{b}} ( {f}_{b 1} , {N}_{t 2} \times u + ntdm ) exp [ - j \frac{2 π {uf}_{s}}{( \frac{{N}_{CB}}{{N}_{t 2}} )} ] & [ 41 ]\end{matrix}$$

For example, in the example of FIG. 12, reception signals of Doppler multiplexed signals of Tx #1 and Tx #3 are included in the output of the ntdm-th Doppler analyzer 211 (ntdm=1), and reception signals of Doppler multiplexed signals of Tx #2 and Tx #3 are included in the output of the ntdm-th Doppler analyzer 211 (ntdm=2).

In addition, multiplex separator 213 obtains a separation signal for each transmission antenna by performing the same separation processing of a Doppler multiplexed signal for each output from the Nₜ₂ Doppler analyzers 211. For example, in the example of FIG. 12, since reception signals of Doppler multiplexed signals of Tx #1 and Tx #3 are included in the output of the ntdm-th Doppler analyzer 211 (ntdm=1), multiplex separator 213 obtains the separation signals of Tx #1 and Tx #3 by performing the separation processing as the Doppler multiplexed signals of N_{DM}=2. Similarly, since reception signals of Doppler multiplexed signals of Tx #2 and Tx #3 are included in the output of the ntdm-th Doppler analyzer 211 (ntdm=2), multiplex separator 213 obtains the separation signals of Tx #2 and Tx #3 by performing the separation processing as the Doppler multiplexed signals of N_{DM}=2.

The subsequent direction estimator 214 and position follower 215 use the reception signal separated for each transmission antenna, and thus have the same operation as that of the above-described embodiment, and can obtain position measurement information using the first transmission frame in the same manner.

As described above, in Variation 4, radar apparatus 1 transmits, in the first transmission frame, a transmission signal for the walking/body movement detection mainly used for the walking/body movement detection and transmits a transmission signal the same as the transmission signal in the second transmission frame by a transmission antenna the same as in the second transmission frame by using a Doppler multiplexed signal different from the transmission signal for the walking/body movement detection. As a result, radar apparatus 1 can obtain position measurement information using the first transmission frame as in the above-described embodiment. In addition, radar apparatus 1 can reduce the power consumption of radar apparatus 1 and can improve the detection accuracy of the vital information (for example, the heartbeat RRI) by performing time multiplexing transmission or simultaneous multiplexing transmission on a transmission signal the same as the second transmission frame in the first transmission frame with a transmission signal for the walking/body movement detection, thereby shortening the interval between the signals for detecting the vital information.

Note that, in the first transmission frame, the transmission of transmission signals for the walking/body movement detection (mainly used for the walking/body movement detection) with the increased number of transmission antennas becomes possible, for example, by increasing the number of Doppler multiplexing. In addition, for example, it is also possible to add code multiplexing to a Doppler multiplexed signal and transmit the signal (coded Doppler multiplexing). The transmission with the further increased number of transmission antennas becomes possible by the coded Doppler multiplexing.

In addition, the example has been described in which a transmission signal for the walking/body movement detection (mainly used for the walking/body movement detection) is subjected to simultaneous multiplexing transmission using the Doppler multiplexing in the first transmission frame, but the present disclosure is not limited thereto, and for example, a transmission signal for the walking/body movement detection may be subjected to time division multiplexing transmission. In this case, in the first transmission frame, a transmission signal for the walking/body movement detection (mainly used for the walking/body movement detection) is subjected to time division multiplexing transmission, and a transmission signal the same as the transmission signal in the second transmission frame can be transmitted by a transmission antenna the same as in the second transmission frame by using a Doppler multiplexed signal different from the transmission signal for the walking/body movement detection. In addition, in this case, radar apparatus 1 can obtain position measurement information using the first transmission frame in the same manner as in the above-described embodiment.

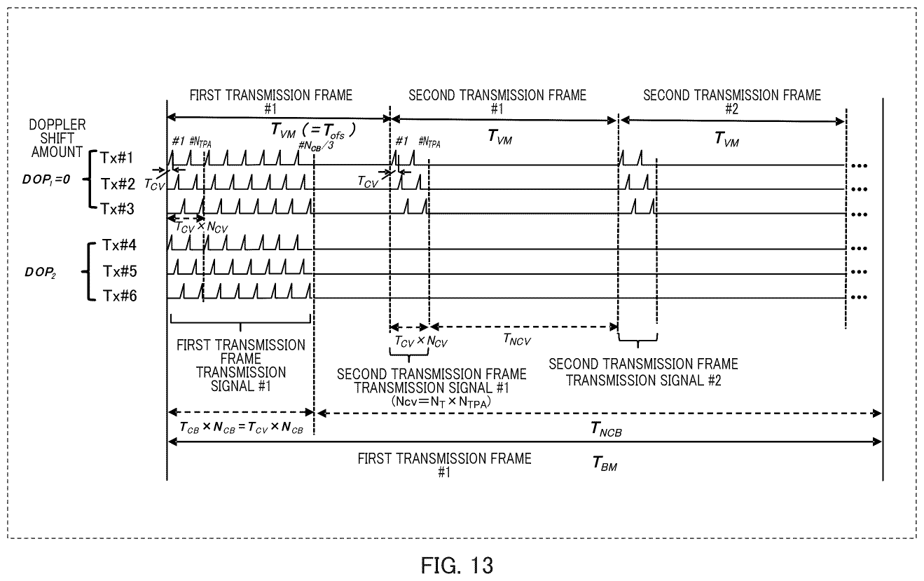

FIG. 13 illustrates an example in which a transmission signal for the walking/body movement detection mainly used for the walking/body movement detection is subjected to time division multiplexing transmission in the first transmission frame. In FIG. 13, the vertical axis for each transmission antenna is a transmission frequency, and a sawtooth-shaped signal portion represents chirp signals. In addition, chirp signals transmitted time-divisionally from the transmission antennas indicate the case where the chirp signals having the same center frequency f_{c} and the same transmission bandwidth B_{w} are transmitted, but the transmitted chirp signals do not need to have the same center frequency f_{c} or the same transmission bandwidth B_{w}. In addition, FIG. 13 illustrates an example in which a transmission signal the same as the transmission signal in the second transmission frame is transmitted by a transmission antenna (Tx #1, 2, 3 in FIG. 13) the same as in the second transmission frame for a period of (T_{CV}×N_{CB}) even after the transmission for a period of (T_{cv}×N_{cv}). As a result, radar apparatus 1 can use a signal continuously transmitted for a period of (T_{CV}×N_{CB}) also as the transmission signal for the walking/body movement detection, so that the number of transmission antennas for the walking/body movement detection can be increased. In the example of FIG. 13, radar apparatus 1 can perform the walking/body movement detection using signals transmitted from the transmission antennas including Tx #1, 2, 3 in addition to Tx #4, 5, 6, so that the detection performance of the walking/body movement detection can be improved.

Here, transmission signals that are transmitted in the first transmission frame and are the same as the transmission signals in the second transmission frame are time-divisionally transmitted from a part of the Nt transmission antennas (Tx #1, 2, 3 in FIG. 13), so that one Doppler shift amount DOP₁ is assigned to these transmission signals. Here, since the Doppler multiplexed phase rotation is not imparted to the transmission signal of the second transmission frame, the Doppler shift amount can be regarded as zero. Therefore, for example, DOP₁=0 may be assigned to a transmission signal that is the same as the transmission signal in the second transmission frame and is transmitted in the first transmission frame in order to impart the same Doppler shift amount. On the other hand, transmission signals for the walking/body movement detection (mainly used for the walking/body movement detection) in the first transmission frame are subjected to time division multiplexing and transmitted from a plurality of transmission antennas (Tx #4, 5, 6 in FIG. 13) using a Doppler shift amount (for example, DOP₁) different from DOP₁ and a multiplexed signal.

The above describes one embodiment according to the present disclosure.

### OTHER EMBODIMENTS

Note that, in the above-described embodiment, the configuration in which a chirp signal is used as the radar transmission signal has been described, but the radar transmission signal may be a signal different from the chirp signal. For example, the radar transmission signal may be a pulse compression wave such as a coded pulse signal. When a coded pulse signal is used as the radar transmission signal, mixer 204 of reception radio 203 converts a high-frequency reception signal into a baseband signal, and uses a correlator (not illustrated) that performs correlation with the coded pulse signal to be transmitted instead of beat frequency analyzer 208, thereby performing the subsequent processing in the same manner as the processing according to each of the above-described embodiments to obtain the same effect.

In addition, a radar apparatus according to the above-described embodiment or variation may be installed indoors or in a vehicle cabin, and the vital detection result output or the position measurement output (information related to the estimation result) from a radar receiver may be used as, for example, sensor information for an indoor surveillance system, health monitoring, or a left-behind detection or a driver's health monitoring in a vehicle cabin. In addition, the radar apparatus may be attached to, for example, a relatively high structure (not illustrated) such as a roadside electric pole or a traffic light. In addition, the radar apparatus may be used as a sensor in, for example, a suspicious person intrusion prevention system (not illustrated). In addition, the position measurement output from the radar receiver may be connected to, for example, a control apparatus (not illustrated) in a driver's health monitoring system or a left-behind detection system in a vehicle cabin, and may be used for alarm call control or abnormality detection control. Note that the application of the radar apparatus is not limited to these, and may be used for other applications. In addition, a target object is a target object to be detected by the radar apparatus, and examples thereof includes vehicles (including four-wheel and two-wheel vehicles), persons, blocks, curbs, and the like. The same applies to the following embodiments.

In addition, in each of the above-described embodiments, the case where the radar apparatus includes vital detector 218 has been described, but the present disclosure is not limited thereto, and, instead of the radar apparatus, an apparatus outside the radar apparatus may include vital detector 218. In this case, the radar apparatus may output the radar position measurement results to the external apparatus.

In a radar apparatus according to an exemplary embodiment of the present disclosure, a radar transmitter and a radar receiver may be individually arranged in physically separate locations from each other. In addition, in a radar receiver according to an exemplary embodiment of the present disclosure, a direction estimator and any other component may be individually arranged in physically separate locations from one another.

In addition, the parameters and numerical values (for example, the number of transmission antennas, the number of reception antennas, the number of code multiplexing, the number of Doppler multiplexing, and parameters related to the first transmission frame and the second transmission frame) used in an embodiment of the present disclosure are examples, and are not limited to these values.

A radar apparatus according to an exemplary embodiment of the present disclosure includes, for example, a central processing unit (CPU), a storage medium such as a read only memory (ROM) that stores a control program, and a work memory such as a random access memory (RAM), which are not illustrated. In this case, the functions of the sections described above are implemented by the CPU executing the control program. However, the hardware configuration of the radar apparatus is not limited to that in this example. For example, the functional sections of the radar apparatus may be implemented as an integrated circuit (IC). Each functional section may be formed as an individual chip, or some or all of them may be formed into a single chip.

Various embodiments have been described with reference to the drawings hereinabove. Obviously, the present disclosure is not limited to these examples. Obviously, a person skilled in the art would arrive at variations and modification examples within a scope described in claims, and it is understood that these variations and modifications are within the technical scope of the present disclosure. The constituent elements of the above-mentioned embodiments may be combined optionally without departing from the spirit of the disclosure.

In addition, any component termed with “processor or section” or with a suffix, such as “-er,” “-or,” or “-ar” in the above-described embodiment may be replaced with “circuit (circuitry),” “device,” “unit,” or “module”.

The above embodiments have been described with an example of a configuration using hardware, but the present disclosure can be realized by software in cooperation with hardware.

Each functional block used in the description of each embodiment described above is typically realized by an LSI, which is an integrated circuit. The integrated circuit controls each functional block used in the description of the above embodiments and may include an input terminal and an output terminal. The LSI may be individually formed as chips, or one chip may be formed so as to include a part or all of the functional blocks. The LSI herein may be referred to as an IC, a system LSI, a super LSI, or an ultra LSI depending on a difference in the degree of integration.

However, the technique of implementing an integrated circuit is not limited to the LSI and may be realized by using a dedicated circuit, a general-purpose processor, or a memory. In addition, a Field Programmable Gate Array (FPGA) that can be programmed after the manufacture of the LSI or a reconfigurable processor in which the connections and the settings of circuit cells disposed inside the LSI can be reconfigured may be used.

If future integrated circuit technology replaces LSIs as a result of the advancement of semiconductor technology or other derivative technology, the functional blocks could be integrated using the future integrated circuit technology. Biotechnology can also be applied.

### Summary of the Present Disclosure

A radar apparatus according to one non-limiting embodiment of the present disclosure includes: control circuitry, which, in operation, controls transmission of a signal by using a first frame and a second frame different from the first frame; and a transmission antenna that transmits the signal, in which
a frame interval of the second frame is shorter than a frame interval of the first frame; a transmission period of the signal in the first frame is shorter than the frame interval of the second frame; the number of transmissions of the signal in the first frame is larger than the number of transmissions of the signal in the second frame; and the first frame includes a transmission duration in which the signal is transmitted and a non-transmission duration in which the signal is not transmitted, and the transmission duration is shorter than the non-transmission duration.

In one non-limiting embodiment of the present disclosure, a plurality of the second frames are set in the non-transmission duration.

In one non-limiting embodiment of the present disclosure, the transmission period of the signal in the first frame is equal to or longer than a transmission period of the signal in the second frame.

In one non-limiting embodiment of the present disclosure, the second frame includes a transmission duration in which the signal is transmitted and a non-transmission duration in which the signal is not transmitted, and the transmission duration is shorter than the non-transmission duration.

In one non-limiting embodiment of the present disclosure, the control circuitry performs simultaneous multiplexing transmission on the signal of the first frame using a plurality of the transmission antennas and performs time multiplexing transmission on the signal of the second frame using a plurality of the transmission antennas.

In one non-limiting embodiment of the present disclosure, the number of the transmission antennas used for the time multiplexing transmission is smaller than a minimum number of transmissions of the signal in the simultaneous multiplexing transmission.

In one non-limiting embodiment of the present disclosure, in the first frame, the control circuitry sets transmission power for each of the plurality of transmission antennas according to the number of the transmission antennas used for the simultaneous multiplexing transmission.

The radar apparatus according to one non-limiting embodiment of the present disclosure further includes: a reception antenna that receives a reflected wave signal, the reflected wave being the signal reflected by an object; first detection circuitry, which, in operation, detects a body movement of the object or performs position measurement of the object by using the reflected wave signal corresponding to the first frame; and second detection circuitry, which, in operation, detects vital information of the object or performs position measurement of the object by using the reflected wave signal corresponding to the second frame.

In one non-limiting embodiment of the present disclosure, the second detection circuitry detects the vital information by using a part of a plurality of the reflected wave signals corresponding to the first frame and the reflected wave signal corresponding to the second frame.

In one non-limiting embodiment of the present disclosure, in the first frame, the control circuitry transmits a first signal common with the signal transmitted in the second frame and a second signal different from the first signal at different timings.

In one non-limiting embodiment of the present disclosure, the control circuitry performs time division multiplexing transmission on the first signal using a plurality of the transmission antennas and performs simultaneous multiplexing transmission on the second signal using a plurality of the transmission antennas.

In one non-limiting embodiment of the present disclosure, in the first frame, the control circuitry performs Doppler multiplexing transmission on a first signal common to the signal transmitted in the second frame and on a second signal different from the first signal using different Doppler shift amounts.

In one non-limiting embodiment of the present disclosure, the control circuitry variably sets the transmission antenna used for the transmission in the second frame and the number of the transmission antennas.

In one non-limiting embodiment of the present disclosure, the control circuitry switches between transmission using the first frame but not the second frame and transmission using the second frame but not the first frame.

In one non-limiting embodiment of the present disclosure, the transmission antenna includes a plurality of antennas; and the control circuitry bundles the plurality of antennas to perform beam transmission on the signal of the first frame or the second frame.

A radar signal processing method according to one non-limiting embodiment of the present disclosure includes: controlling transmission of a signal by using a first frame and a second frame different from the first frame; and transmitting the signal, in which
a frame interval of the second frame is shorter than a frame interval of the first frame; a transmission period of the signal in the first frame is shorter than the frame interval of the second frame; the number of transmissions of the signal in the first frame is larger than the number of transmissions of the signal in the second frame; and the first frame includes a transmission duration in which the signal is transmitted and a non-transmission duration in which the signal is not transmitted, and the transmission duration is shorter than the non-transmission duration.

A vital detection apparatus according to one non-limiting embodiment of the present disclosure includes: control circuitry, which, in operation, controls transmission of a signal by using a first frame and a second frame different from the first frame; a transmission antenna that transmits the signal; a reception antenna that receives a reflected wave signal, the reflected wave being the signal reflected by a target object; and detection circuitry, which, in operation, detects vital information based on the reflected wave signal, in which
a frame interval of the second frame is shorter than a frame interval of the first frame; a transmission period of the signal in the first frame is shorter than the frame interval of the second frame; the number of transmissions of the signal in the first frame is larger than the number of transmissions of the signal in the second frame; and the first frame includes a transmission duration in which the signal is transmitted and a non-transmission duration in which the signal is not transmitted, and the transmission duration is shorter than the non-transmission duration.

Radar signal processing circuitry according to one non-limiting embodiment of the present disclosure includes: signal generation circuitry, which, in operation, generates a first frame and a second frame different from the first frame; and control circuitry, which, in operation, controls a signal transmitted via a transmission antenna by using the first frame and the second frame, in which
a frame interval of the second frame is shorter than a frame interval of the first frame; a transmission period of the signal in the first frame is shorter than the frame interval of the second frame; the number of transmissions of the signal in the first frame is larger than the number of transmissions of the signal in the second frame; and the first frame includes a transmission duration in which the signal is transmitted and a non-transmission duration in which the signal is not transmitted, and the transmission duration is shorter than the non-transmission duration.

While various embodiments have been described herein above, it is to be appreciated that various changes in form and detail may be made without departing from the sprit and scope of the disclosure(s) presently or hereafter claimed.

This application is entitled to and claims the benefit of Japanese Patent Application No. 2025-041394, filed on Mar. 14, 2025, the disclosure of which including the specification, drawings and abstract is incorporated herein by reference in its entirety.

### INDUSTRIAL APPLICABILITY

The present disclosure is suitable as a radar apparatus that detects a wide angle range.

### REFERENCE SIGNS LIST

1 Radar apparatus
100, 100a, 100b Radar transmitter
101 Radar transmission signal generator
102, 102a, 102b Signal controller
103 Modulation signal generator
104 VCO
105, 105a, 105b Transmission controller
106 Phase rotator
107 Switch
108 Transmission antenna section
109 Transmission antenna selection controller
110 Transmission frame adaptive controller
200, 200c Radar receiver
201 Antenna system processor
202 Reception antenna section
203 Reception radio
204 Mixer
205 LPF
206 Signal processor
207 A/D converter
208 Beat frequency analyzer
209 Output switch
210 Storage
211 Doppler analyzer
212 CFAR section
213 Multiplex separator
214 Direction estimator
215 Position follower
216 Signal extractor
217, 217c Beam-weight multiplier
218, 218c Vital detector
219 Separation extractor

1. A radar apparatus, comprising:
control circuitry, which, in operation, controls transmission of a signal by using a first frame and a second frame different from the first frame; and
a transmission antenna, which, in operation, transmits the signal, wherein
a frame interval of the second frame is shorter than a frame interval of the first frame,
a transmission period of the signal in the first frame is shorter than the frame interval of the second frame,
the number of transmissions of the signal in the first frame is larger than the number of transmissions of the signal in the second frame, and
the first frame includes a transmission duration in which the signal is transmitted and a non-transmission duration in which the signal is not transmitted, and the transmission duration is shorter than the non-transmission duration.

2. The radar apparatus according to claim 1, wherein
a plurality of the second frames are set in the non-transmission duration.

3. The radar apparatus according to claim 1, wherein
the transmission period of the signal in the first frame is equal to or longer than a transmission period of the signal in the second frame.

4. The radar apparatus according to claim 1, wherein
the second frame includes a transmission duration in which the signal is transmitted and a non-transmission duration in which the signal is not transmitted, and the transmission duration is shorter than the non-transmission duration.

5. The radar apparatus according to claim 1, wherein
the control circuitry performs simultaneous multiplexing transmission on the signal of the first frame using a plurality of the transmission antennas and performs time multiplexing transmission on the signal of the second frame using a plurality of the transmission antennas.

6. The radar apparatus according to claim 5, wherein
the number of the transmission antennas used for the time multiplexing transmission is smaller than a minimum number of transmissions of the signal in the simultaneous multiplexing transmission.

7. The radar apparatus according to claim 5, wherein
in the first frame, the control circuitry sets transmission power for each of the plurality of transmission antennas according to the number of the transmission antennas used for the simultaneous multiplexing transmission.

8. The radar apparatus according to claim 1, further comprising:
a reception antenna, which, in operation, receives a reflected wave signal, the reflected wave being the signal reflected by an object;
first detection circuitry, which, in operation, detects a body movement of the object or performs position measurement of the object by using the reflected wave signal corresponding to the first frame; and
second detection circuitry, which, in operation, detects vital information of the object or performs position measurement of the object by using the reflected wave signal corresponding to the second frame.

9. The radar apparatus according to claim 8, wherein
the second detection circuitry detects the vital information by using a part of a plurality of the reflected wave signals corresponding to the first frame and the reflected wave signal corresponding to the second frame.

10. The radar apparatus according to claim 9, wherein
in the first frame, the control circuitry transmits a first signal common with the signal transmitted in the second frame and a second signal different from the first signal at different timings.

11. The radar apparatus according to claim 10, wherein
the control circuitry performs time division multiplexing transmission on the first signal using a plurality of the transmission antennas and performs simultaneous multiplexing transmission on the second signal using a plurality of the transmission antennas.

12. The radar apparatus according to claim 9, wherein
in the first frame, the control circuitry performs Doppler multiplexing transmission on a first signal common to the signal transmitted in the second frame and on a second signal different from the first signal using different Doppler shift amounts.

13. The radar apparatus according to claim 1, wherein
the control circuitry variably sets the transmission antenna used for the transmission in the second frame and the number of the transmission antennas.

14. The radar apparatus according to claim 1, wherein
the control circuitry switches between transmission using the first frame but not the second frame and transmission using the second frame but not the first frame.

15. The radar apparatus according to claim 1, wherein:
the transmission antenna includes a plurality of antennas; and
the control circuitry bundles the plurality of antennas to perform beam transmission on the signal of the first frame or the second frame.

16. A radar signal processing method, comprising:
controlling transmission of a signal by using a first frame and a second frame different from the first frame; and
transmitting the signal, wherein
a frame interval of the second frame is shorter than a frame interval of the first frame,
a transmission period of the signal in the first frame is shorter than the frame interval of the second frame,
the number of transmissions of the signal in the first frame is larger than the number of transmissions of the signal in the second frame, and
the first frame includes a transmission duration in which the signal is transmitted and a non-transmission duration in which the signal is not transmitted, and the transmission duration is shorter than the non-transmission duration.

17. A vital detection apparatus, comprising:
control circuitry, which, in operation, controls transmission of a signal by using a first frame and a second frame different from the first frame;
a transmission antenna, which, in operation, transmits the signal;
a reception antenna that receives a reflected wave signal, the reflected wave being the signal reflected by a target object; and
detection circuitry, which, in operation, detects vital information based on the reflected wave signal, wherein
a frame interval of the second frame is shorter than a frame interval of the first frame,
a transmission period of the signal in the first frame is shorter than the frame interval of the second frame,
the number of transmissions of the signal in the first frame is larger than the number of transmissions of the signal in the second frame, and
the first frame includes a transmission duration in which the signal is transmitted and a non-transmission duration in which the signal is not transmitted, and the transmission duration is shorter than the non-transmission duration.

18. Radar signal processing circuitry, comprising:
signal generation circuitry, which, in operation, generates a first frame and a second frame different from the first frame; and
control circuitry, which, in operation, controls a signal transmitted via a transmission antenna by using the first frame and the second frame, wherein
a frame interval of the second frame is shorter than a frame interval of the first frame,
a transmission period of the signal in the first frame is shorter than the frame interval of the second frame,
the number of transmissions of the signal in the first frame is larger than the number of transmissions of the signal in the second frame, and
the first frame includes a transmission duration in which the signal is transmitted and a non-transmission duration in which the signal is not transmitted, and the transmission duration is shorter than the non-transmission duration.
